(19) United States
(12) Patent Application Publication
(10) Pub. No.: US 2024/0342090 A1
(43) Pub. Date: Oct. 17, 2024

(54) METHOD OF APPLYING ATOMICALLY THIN LAYERS OF METAL OXIDES ON ACTIVE PHARMACEUTICAL INGREDIENTS FOR THEIR DELAYED AND TARGETED RELEASE AND ACTIVE PHARMACEUTICAL INGREDIENTS PREPARED BY SAID METHOD

(71) Applicant: University of Central Florida Research Foundation, Inc., Orlando, FL (US)

(72) Inventors: Parag Banerjee, Orlando, FL (US); Jaynlynn Sosa, Orlando, FL (US)

(73) Assignee: University of Central Florida Research Foundation, Inc., Orlando, FL (US)

(21) Appl. No.: 18/443,496

(22) Filed: Feb. 16, 2024

Related U.S. Application Data
(60) Provisional application No. 63/459,784, filed on Apr. 17, 2023.

(51) Int. Cl. A61K 9/16; A61K 31/192

(52) U.S. Cl. CPC A61K 9/1611; A61K 9/1682; A61K 31/192

(57) ABSTRACT

A method to coat active pharmaceutical ingredient (API) powders with atomically thin layers of biocompatible metal oxide films such as aluminum oxide and zinc oxide films. Metal oxide films provide a barrier that controllably dissolves and releases the active pharmaceutical ingredients (APIs) in different pH environments such as is found in the human gastrointestinal (GI) tract. Coated API powders, can be prepared using the described methods, such as 5-Aminosalicylic acid (5-ASA) powder coated with aluminum oxide and zinc oxide films.

### STATEMENT OF FEDERALLY SPONSORED RESEARCH

This invention was made with government support under NSF-1562102 & 1908167 awarded by the National Science Foundation (NSF) and AFOSR-FA9550-19-1-0127 awarded by the Air Force Office of Scientific Research (AFOSR). The U.S. government has certain rights to the invention.

### FIELD OF THE INVENTION

This invention generally relates to using atomic layer deposition (ALD) technology in biomedical applications such as applying coatings to drugs for targeted and/or controllable release, particularly to coating active pharmaceutical ingredient (API) powders with metal oxides using ALD technology, and most particularly to coating 5-aminosalicyclic acid (5-ASA) powder with aluminium oxide (Al₂O₃) and/or zinc oxide (ZnO) using ALD technology.

### BACKGROUND

### Powder Atomic Layer Deposition (ALD) for Biomedical Applications

The first applications of atomic layer deposition (ALD) were limited to microelectronics, semiconductors, capacitors, and interconnects. However, in recent years, film depositions that show promising effects in biologically relevant substrates have been developed¹.

ALD has been shown to provide surface and chemical stability on pharmaceutical powders to improve their processing and flowability. For instance, powder substrates with a wider variety of physicochemical characteristics such as dibasic calcium phosphate dihydrate (DCPD), Sorbitol (SORB), α-lactose monohydrate (LAC), microcrystalline cellulose (MCC), and Croscarmellose-sodium (Na—CC) have been coated with ALD to show the effect of thin film deposition on pharmaceutical powders 2. Hirschberg et al., show that as little as 5 layers of titanium dioxide (TiO₂) reduced electrostatic interactions between particles and facilitated secondary powder processing (FIG. 1A)².

Film coating via ALD has also played a part in coating nanoparticles used as contrast agents for bioimaging and disease diagnosis. Powder ALD has been reported to improve the magnetic stability of magnetic resonance imaging (MRI) contrast agents such as FDA approved iron oxides (Fe₂O₃) typically used for T2 weighted MRIs. Duan et al.,³ reported that uncoated Fe₂O₃ particles are easily oxidized in ambient conditions and benefit from a 5 nm thick aluminium oxide (Al₂O₃) layer deposited by ALD. The thin film passivates oxygen exposure and prevents the loss of high contrast MRI capabilities as shown in FIGS. 1B-C, via thermogravimetric analysis and saturation magnetization studies.

Other studies have also taken advantage of ALD films to improve the magnetic properties of metallic nickel nanoparticles and achieve targeted drug delivery. Uudeküll et al., used TiO₂ film deposition on generally cytotoxic metallic nickel particles for targeted drug delivery to improve their magnetization (FIG. 1D). ALD has also been implemented to potentially improve dental implant materials. For example, ALD on silica nanoparticles using FBRs have been successfully coated with Al₂O₃ ALD and could be used for orthodontic applications⁴ (FIG. 1E).

In addition to biomedical applications such as improving pharmaceutical powder mechanical properties, diagnostic imaging, and targeted drug delivery, ALD is increasingly being used for applications in delayed release of drugs and vaccines.

### Powder Atomic Layer Deposition (ALD) for Delayed Drug Release

ALD has made successful forays into the biomedical industry, and there is now an expanding interest for particles coated with ALD for delayed release. Coating irregularly shaped pharmaceutical powders not only improves processability, loading efficiency, and stability of solid drugs, but also has enormous potential in delaying drug release times for better patient compliance and long-term treatment plans. Expanding the ability of ALD to coat small particles can also reduce the harsh side effects seen in pharmaceuticals for acute and chronic disease.

Kääriäinen et al., have studied the effect of aluminium oxide (Al₂O₃), zinc oxide (ZnO), and titanium dioxide (TiO₂) ALD coatings on acetaminophen powders⁵. Out of these, TiO₂ films on acetaminophen were the most effective in being the least cytotoxic to human epithelial colorectal adenocarcinoma Caco-2 cell lines while still reducing the drug release % of acetaminophen. The release of acetaminophen coated with Al₂O₃, ZnO, and TiO₂ was then studied in phosphate buffer saline (PBS) solutions of pH 6.8, and in acidic buffer solutions of pH 1.2. Their results show that uncoated powders dissolved readily in both solutions (2 minutes) (FIGS. 2A-B, FIG. 3). In contrast, coated powders released the active pharmaceutical drug after longer periods of time ranging from 20 minutes for Al₂O₃ and TiO₂ in 1.2 pH, and up to 6 hours for ZnO in 6.8 pH. Coating acetaminophen with ALD films enhanced the powder's surface stability and could potentially allow for easier pharmaceutical processability into tablets, injections, pellets, and capsules.

Multifunctional nanoparticles such as iron oxide (Fe₂O₃), typically used for medical treatment and diagnosis, have shown promising results with zinc oxide (ZnO) ALD film coatings. In fact, Seong et al., coated superparamagnetic ibuprofen loaded Fe₃O₄ with 100 nm of ZnO ALD⁶. The ZnO film did not affect the magnetic properties of the Fe₃O₄ nanoparticle core, while sustained release of ibuprofen was obtained for up to 72 hours (FIG. 2C).

Hellrup et al., sustained drug release profiles for up to 12 weeks using indomethacin as a model drug⁷. In this case, indomethacin, which is an anti-inflammatory drug normally prescribed to treat acute pain, was coated with 115 cycles of Al₂O₃ ALD. The final thickness of the Al₂O₃ film was 30 nm, which is attributed to a higher than usual Al₂O₃ ALD growth rate. To test the sustained drug release, different drug doses of 1, 10, and 100 mg/kg were injected into rats. Rat blood plasma was collected after 1, 2, 4, 6, 8, 10, and 12 weeks to quantify the concentration of indomethacin. The higher drug doses of 10 and 100 mg/kg were released for up to 12 weeks, whereas the smaller drug dose of 1 mg/kg reached the lower limit of concentration at week 10 (FIG. 2D, FIG. 3). Nevertheless, the Al₂O₃ ALD nanoshell delayed indomethacin's release over several weeks in comparison to previous reports.

Similarly, Zhang et al. proved the feasibility of ALD to increase the release time of active drug powders in the nanometer to micrometer range. Budesonide and lactose particles were coated with Al₂O₃ ALD⁸. Unlike other reports, this study focused on determining the minimum number of cycles needed to alter the dissolution rate of budesonide and lactose in biological media. It was reported that 2-4 cycles are required to delay drug release and achieve less particle agglomeration for better powder processability (FIGS. 2E-G, FIG. 3).

Further work on budesonide particles has been conducted by D. La Zara et al., to increase particle lung retention and deposition for longer lasting chronic obstructive pulmonary disease (COPD) and asthma inhaled treatments⁹. Multiple ALD nanofilms including SiO₂, Al₂O₃, and TiO₂ were tested. Results showed that coating the budesonide particles with any of the ALD films tested, lead to better aerosolization of budesonide. The ALD coatings decreased van der Waals interactions between particles and prevented their agglomeration at the pulmonary target sites. They reported that a minimum of 10 nm thick coatings of either SiO₂, Al₂O₃, and TiO₂ ALD films are ideal to improve drug retention and delayed delivery at the lungs of perfused mice (FIG. 2H, FIG. 3).

Garcea et al., disclosed a ground-breaking use of ALD for coating human papilloma virus vaccine powders containing HPV16 L1 capsomeres¹⁰. This study proved ALD's ability to control the release of antibody titers depending on the number of monolayers used to coat the powders. A lower number of Al₂O₃ monolayers released antibodies more quickly than powders that were coated with more monolayers (100-500 cycles of Al₂O₃ ALD were tested) (FIG. 2I). The Al₂O₃ ALD coating did not only show promising results in kinetics, but also in protecting the vaccines from harsh environmental conditions, such as prolonged storing time at temperatures of up to 50° C.

Despite these notable advances made in drug release systems, there is still no single system available in the market with which both targeted and delayed release can be achieved. Availability of such a drug release system, capable of both targeted and delayed release, would greatly improve therapeutic options for treatment of disease, particularly in chronic illnesses that often require long term treatment.

### SUMMARY OF THE INVENTION

The invention develops a methodology to coat active pharmaceutical ingredient (API) powders with atomically thin layers of biocompatible metal oxide films such as aluminum oxide. Metal oxide films provide a barrier that controllably dissolves and releases the active pharmaceutical ingredients (APIs) in different pH environments such as is found in the human gastrointestinal (GI) tract. The thickness of the film, only a few tens of nanometers, and the precise atomic nanoscale engineering to control the composition and hence, film dissolution rate, provides an entirely new approach to develop API release technology which is highly controllable, less bulky as compared to current enteric formulations in standard use and can lead to increased potency per unit volume of an ingested tablet. Further, fine tuning the method to respond to the specific chemistry of the GI tract can lead to highly targeted drug release, or delayed drug release over time. Taken together, these advantages can lead to better patient outcomes. Therefore, this technology brings conventional methods a step closer to customizable drug delivery for personalized medicine.

To overcome the challenges of the drug release systems mentioned above in the Background section, this invention proposes the synthesis of a single drug release system in which both targeted and delayed release can be achieved.

In a general aspect, the invention provides a new application for atomic layer deposition (ALD) technology.

In a general aspect, the invention provides a drug coating that can be manipulated to produce desired properties that improve and/or enhance drug delivery.

In another aspect, the invention provides a new paradigm for drug delivery. The inventive method enables both targeted and delayed release of a drug using a single drug delivery system. Such new methods may decrease the amount of drug required for effective treatment and/or decrease the number of times a patient needs to take the drug. These features are particularly advantageous in chronic illnesses that often require long term treatment.

In an embodiment, the invention provides a method for coating powder particles with metal oxides, particularly biocompatible metal oxides. Non-limiting examples of such metal oxides include aluminium oxide (Al₂O₃) and zinc oxide (ZnO). A tool that performs atomic layer deposition (ALD), an ALD reactor/system, can be used to carry out the method. Non-limiting examples include a powder reactor such as a rotary reactor, a fluidized bed reactor, and a continuous coating reactor. General information regarding ALD reactors was obtained from the website of the company Beneq on Feb. 9, 2023.

In an embodiment, the invention provides a method for coating powder particles with metal oxide. This method encompasses sequentially carried out steps including loading a predetermined amount of powder particles into a rotary barrel reactor of a rotary thermal atomic layer deposition (ALD) system; activating a motor of the rotary thermal ALD system to rotate the rotary barrel reactor such that the powder particles are in motion when carrying out the method/when implementing steps of the method; submitting the powder particles to at least one cycle of an ALD process, thereby coating the powder particles with the metal oxide to produce coated powder particles; and unloading the coated powder particles from the rotary thermal ALD system upon completion of the at least one cycle of the ALD process. The powder particles must stay in motion as the method is implemented to assure coating of the three-dimensional surfaces of the powder particles.

It should be understood that although the steps of the methods set forth herein can be carried out sequentially such steps are not necessarily required to be performed in the order described, and the order of the steps of such methods should be understood to be merely exemplary. Likewise, additional steps may be included in such methods, and certain steps may be omitted or combined in methods consistent with various embodiments of the present methods and/or compositions.

Additionally, although the elements in the following method claims, if any, are recited in a particular sequence with corresponding labeling (unless the claim recitations otherwise imply a particular sequence for implementing some or all of those elements), those elements are not necessarily intended to be limited to being implemented in that particular sequence.

The inventive method is contemplated for use with powdered particles of any material. In an embodiment, the powder particles are of a small molecule having a pharmaceutical use for treatment, diagnosis, and/or prevention of disease. In a preferred, albeit non-limiting example, the powder particles can be powder particles of an active pharmaceutical ingredient (API). An “active pharmaceutical ingredient” refers to an ingredient of a drug that is responsible for the desired effect of the drug. A non-limiting example of an active pharmaceutical ingredient is 5-Aminosalicylic acid (5-ASA).

In an embodiment of the method, one cycle of an atomic layer deposition (ALD) process includes a series of steps. The steps include subjecting the powder particles to one pulse of a gaseous precursor metal, such as (but not limited to), trimethyl aluminium (TMA) and diethyl zinc (DEZ); subjecting the powder particles to a first purge of argon gas; subjecting the powder particles to one pulse of deionized water; and completing the at least one cycle of the ALD process by subjecting the powder particles to a second purge of the argon gas. The parameters of each step of the series of steps can be optimized for desired outcome. For example, one pulse of a gaseous precursor metal can be, but is not limited to, a time of one second; one pulse of a deionized water can be, but is not limited to, a time of 0.5 second; and first and second purges of argon gas can be, but are not limited to, 20 second flows of argon at 75 standard cubic centimetre per minute (sccm).

In an embodiment of the method, the at least one cycle of the ALD process is carried out at a temperature of less than 150°, preferably, but not limited to, at a temperature of 120° C.

In another embodiment of the method, the at least one cycle of the ALD process is followed by additional sequential cycles of the ALD process. For example, the number of additional ALD cycles carried out can be between one additional cycle and 300 additional cycles. Further, non-limiting examples are an ALD process of 200 cycles or an ALD process of 300 cycles.

In another embodiment, the invention provides powder particles having a coating of metal oxide prepared according to the methods described herein. The metal oxide coating can be composed of aluminium oxide (Al₂O₃) or zinc oxide (ZnO). The coating of metal oxide can be formed in a range of thicknesses, such as, but not limited to, a thickness corresponding to a thickness on a planar silicon substrate in a range of 30 to 33 nm. The coating of metal oxide can be conformational on the surface of the powder particles. A non-limiting example of these powder particles is 5-Aminosalicylic acid (5-ASA) powder particles coated with aluminum oxide or zinc oxide. 5-Aminosalicylic acid is an anti-inflammatory drug used often for treatment of chronic illnesses.

In another embodiment, the invention provides a method for treating a gastrointestinal condition, such as, but not limited to, inflammatory bowel disease (IBD), in a subject in need thereof. The method includes providing a pharmaceutical composition including 5-Aminosalicylic acid (5-ASA) powder coated with a metal oxide and administering a pharmaceutically effective amount of the pharmaceutical composition to the subject, thereby treating the gastrointestinal condition in the subject. Non-limiting examples of inflammatory bowel disease (IBD) are Crohn's Disease and ulcerative colitis.

The term “subject” refers to any human or animal who will benefit from use of the coated powder particles, compositions, methods, and/or treatments described herein.

The phrases “effective amount”, “pharmaceutically effective amount” and “therapeutically effective amount” refer to the amount of a composition necessary to achieve the composition's intended function.

Unless explicitly stated otherwise, each numerical value and range should be interpreted as being approximate as if the word “about” or “approximately” preceded the value of the value or range.

Additionally, the term “or” is intended to mean an inclusive “or” rather than an exclusive “or.” That is, unless specified otherwise or clear from context, “X employs A or B” is intended to mean any of the natural inclusive permutations. That is if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present invention may be obtained by references to the data shown in the accompanying drawings when considered in conjunction with the subsequent detailed description. Any embodiments illustrated in the drawings are intended only to exemplify the invention and should not be construed as limiting the invention to the illustrated embodiments.

FIGS. 1A-E show data from the background art regarding powder atomic layer deposition (ALD) for biomedical applications.

FIG. 1A is a bar graph showing that 5 monolayers of titanium dioxide (TiO₂) improve flow rate of DCPD, SORB, ALC, MCC, and Na—CC powders, adapted from Hirschberg² et al., 2019.

FIG. 1B is a graph showing that ALD coating prevents mass gain of iron trioxide (Fe₂O₃) particles upon oxidation, adapted from Duan³ et al., 2016.

FIG. 1C is a graph showing that ALD coating improves magnetization of iron trioxide (Fe₂O₃) particles, adapted from Duan³ et al., 2016.

FIG. 1D is a graph showing that TiO₂ ALD coating improves magnetization of metallic nickel nanoparticles, adapted from Uudeküll et al., 2017.

FIG. 1E is a micrograph of a silica nanoparticle coated with aluminum oxide (Al₂O₃), adapted from Hakim⁴ et al., 2005.

FIGS. 2A-I show data from the background art regarding powder atomic layer deposition (ALD) for delayed drug release.

FIG. 2A is a graph showing delayed release of acetaminophen (ACTM) in pH 6.8, adapted from Kääriäinen⁵ et al., 2017.

FIG. 2B is a graph showing delayed release of acetaminophen (ACTM) in pH 1.2, adapted from Kääriäinen⁵ et al., 2017.

FIG. 2C is a graph showing ibuprofen release over time (hours), adapted from Seong⁶ et al., 2019.

FIG. 2D is a graph showing indomethacin concentration (ng/ml) in blood plasma over time (weeks), adapted from Hellrup⁷ et al., 2019.

FIG. 2E is a graph showing delayed release of aluminum oxide (Al₂O₃) ALD coated lactose particles, adapted from Zhang⁸ et al., 2017.

FIG. 2F is a graph showing delayed release of aluminum oxide (Al₂O₃) ALD coated budesonide particles, adapted from Zhang⁸ et al., 2017.

FIG. 2G is a micrograph of a budesonide particle coated with aluminum oxide (Al₂O₃, alumina) ALD, adapted from Zhang⁸ et al., 2017.

FIG. 2H is a graph showing release of budesonide particles from films coated with SiO₂, TiO₂, and Al₂O₃ ALD, adapted from D. La Zara⁹ et al., 2021.

FIG. 2I is a graph showing anti-HPV 16 titer release over days, adapted from Garcea¹⁰ et al., 2020.

FIG. 3 shows the chemical/molecular structures of budesonide, lactose, indomethacin, and acetaminophen which were coated using atomic layer deposition (ALD) for experimental delayed drug release (Kääriäinen⁵ et al., 2017, Zhang⁸ et al., 2017, Hellrup⁷ et al., 2019, and D. La Zara⁹ et al., 2021).

FIG. 4 shows the chemical/molecular structures classes of aminosalicylates: 5-aminosalicyclic acid (5-ASA), sulfasalazine, balsalazide, and olsalazine, adapted from Campreher¹⁹ et al., 2011.

FIG. 5 is a schematic illustration of the gastrointestinal (GI) tract showing pathology of both types of inflammatory bowel disease (IBD): Crohn's disease (CD) on the right and ulcerative colitis (UC) on the left.

FIGS. 6A-B show 5-aminosalicyclic acid (5-ASA) delayed and targeted release in the gastrointestinal (GI) tract.

FIG. 6A is a graph showing 5-ASA release over a period of time and at different pHs.

FIG. 6B is a schematic illustration of the gastrointestinal (GI) tract showing both types of inflammatory bowel disease (IBD): Crohn's disease (CD) on the left and ulcerative colitis (UC) on the right. Differences in intestinal pH are indicated. In CD, the caecum is indicated having a pH of ˜5. In UC, the descending colon is indicated having a pH of ˜7.

FIG. 7 is a graph showing cumulative % of release of Pentasa® and Asacol® over time. FIG. 7 also shows photos of Pentasa® and Asacol®; Pentasa® on the left side having an ethylcellulose coating and Asacol® on the right side having an acrylic resin coating.

FIG. 8 is a schematic illustration of the experimental methodology proposed by the instant invention.

FIGS. 9A-C show data resulting from carrying out attenuated total reflectance-Fourier-transformed infrared (FTIR) spectroscopy of coated and uncoated powders.

FIG. 9A shows an ATR FTIR spectrum of control 5-ASA (uncoated), 300CyAl₂O₃@5-ASA, and 200CyZnO@5-ASA powders. For the Al₂O₃ coated powder, the broad peak at 3633 cm⁻¹ is identified as OH-stretching, while the boxed region (850-1050 cm⁻¹) shows the Al—O bonding.

FIG. 9B shows Thermogravimetric analysis (TGA) data of control (uncoated), 300CyAl₂O₃@5-ASA, and 200CyZnO@5-ASA. The final ceramic yield is labeled as ‘8’. Inset shows a histogram plot of 8.

FIG. 9C shows an ATR FTIR spectrum of control 5-ASA (uncoated) and 300CyAl₂O₃@5-ASA powders. This ATR FTIR spectrum shows that the primary peaks (molecular vibrations) of the control (uncoated powder) and of the coated powder remain intact, except for the OH showing dihydroxylation of the OH group so that the Al₂O₃ can anchor on the 5-ASA surface.

FIG. 10A shows an optical image of control 5-ASA (uncoated).

FIG. 10B shows an optical image of 300CyAl₂O₃@5-ASA.

FIG. 10C shows an optical image of 200CyZnO@5-ASA.

FIG. 10D shows a scanning electron microscopy (SEM) image of control 5-ASA

(uncoated).

FIG. 10E shows an SEM image of 300CyAl₂O₃@5-ASA.

FIG. 10F shows an SEM image of 200CyZnO@5-ASA.

FIG. 10G shows an energy dispersive spectrum (EDS) spectrum of control 5-ASA

(uncoated).

FIG. 10H shows an EDS spectrum of 300CyAl₂O₃@5-ASA.

FIG. 10I shows an EDS spectrum of 200CyZnO@5-ASA.

FIG. 11A shows a low magnification Bright-Field Transmission Electron Microscopy (BF-TEM) image of 300CyAl₂O₃@5-ASA.

FIG. 11B shows a high magnification BF-TEM image from the box in FIG. 11A showing the conformal nature of the Al₂O₃ ALD film with a shell thickness of 38.0±1.2 nm.

FIG. 11C shows a high-angle annular dark field-scanning transmission electron microscopy (HAADF-STEM) image of 300CyAl₂O₃@5-ASA particle. The box shows the region mapped for EDS analysis.

FIG. 11D shows STEM-EDS mapping of 300CyAl₂O₃@5-ASA showing aluminum (Al) elemental map indicating the presence of Al₂O₃ on the surface of 5-ASA.

FIG. 11E shows STEM-EDS mapping of 300CyAl₂O₃@5-ASA showing carbon (C) signal.

FIG. 11F shows STEM-EDS mapping of 300CyAl₂O₃@5-ASA showing oxygen (O) elemental map.

FIG. 12A shows a high-resolution transmission electron microscopy (HR-TEM) image of 200CyZnO@5-ASA. The ZnO shell thickness is 24.7±2.8 nm.

FIG. 12B shows a HAADF STEM image of 200CyZnO@5-ASA.

FIG. 12C shows STEM-EDS mapping of 200CyZnO@5-ASA showing zinc (Zn) elemental map indicating the presence of ZnO on the surface of 5-ASA.

FIG. 12D shows STEM-EDS mapping of 200CyZnO@5-ASA showing carbon (C) signal.

FIG. 12E shows STEM-EDS mapping of 200CyZnO@5-ASA showing oxygen (O) elemental map.

FIG. 13A shows a quartz crystal microbalance (QCM) plot showing mass loss in μg/cm² of 30 nm of Al₂O₃ in pH 4 HCl solution. Inset on bottom right shows a typical QCM crystal with a well-defined Au electrode, further coated with ALD.

FIG. 13B shows a QCM plot showing mass loss in μg/cm² of 27 nm of ZnO in pH 4 HCl solution.

FIG. 14 shows a representative UV-vis spectrum of a 70 μg/mL concentration of 5-ASA in HCl solution.

FIG. 15A shows release rates of control 5-ASA and 300CyAl₂O₃@5-ASA at 400 rpm.

FIG. 15B shows release rates of control 5-ASA and 300CyAl₂O₃@5-ASA at 23 rpm.

FIG. 15C shows release rates of control 5-ASA and 300CyAl₂O₃@5-ASA at 0 rpm (no stirring). *For FIGS. 15A-C data was obtained by averaging a set of three runs per sample and the error associated is shown as filled area in the background. Solid lines show fit based on Equation 1:

$$abs ( t ) = \frac{{P}_{1} t}{{P}_{2} + t} .$$

FIG. 16A shows release rate studies under no stirring conditions of control 5-ASA and 300CyAl₂O₃@5-ASA.

FIG. 16B shows release rate studies under no stirring conditions of control 5-ASA and 200CyZnO@5-ASA. *For FIGS. 16A-B solid lines show fits based on Equation 1.

FIGS. 17A-C show release rates of uncoated and coated powders in different form factors at pH 4. The release rates are delayed due to the ALD coating. All powders were tested at pH 4 (acidic).

FIG. 17A shows the release rates of the 5-ASA as a pellet before and after coating with 300 cycles of ALD Al₂O₃.

FIG. 17B shows the release rates of 5-ASA as a film on a substrate before and after coating with 300 cycles of ALD Al₂O₃.

FIG. 17C shows the release rate of 5-ASA as a free powder before and after coating with 300 cycles of ALD Al₂O₃.

FIGS. 18A-C show release rates of uncoated and coated powders resulting from stirring or lack thereof (with 300 cycles of ALD Al₂O₃). This data shows that 5-ASA particle breakage can lead to dissolution of the drug during vigorous stirring.

FIG. 18A shows fast stirring at 400 rpm.

FIG. 18B shows slow stirring at 23 rpm.

FIG. 18C shows no stirring.

FIGS. 19A-C show release rates of ZnO coated 5-ASA.

FIG. 19A shows fast stirring at 400 rpm.

FIG. 19B shows slow stirring at 23 rpm.

FIG. 19C shows no stirring.

FIGS. 20A-D show release rate of unstirred 5-ASA powder, uncoated, ALD Al₂O₃ and ALD ZnO in pH 4 (acidic). Both Al₂O₃ and ZnO are categorized as fully biocompatible metal oxides that have no side effects when ingested. However, while Al₂O₃ is stable in pH 4 solution, ZnO starts to dissolve immediately.

FIG. 20A shows the rate of drug release with no coating (grey) and with 300 cycles of ALD Al₂O₃ (30 nm in thickness and shown in red). A clear comparison of the effectiveness of the ALD coating is shown. Even after 5000 seconds (i.e., 1 hour and 24 minutes) the coated sample is unable to release the 5-ASA.

FIG. 20B shows quartz crystal microbalance. To understand the result highlighted in FIG. 20A, quartz crystal microbalance result shows that Al₂O₃ coated quartz crystal shows negligible mass loss when immersed in a pH 4 solution.

FIG. 20C shows that 5-ASA coated with 200 cycles ALD ZnO (˜28 nm in thickness and shown in blue) starts to dissolve in pH 4 solution. Once completely dissolved, the 5-ASA is released into solution after around 1000 seconds (i.e., 17 minutes).

FIG. 20D shows quartz crystal microbalance data that shows immediate mass loss of a quartz crystal coated with 28 nm ZnO film upon immersing in a pH 4 solution.

FIG. 21 is a graph showing the FTIR spectrum of 5-aminosalicyclic acid (5-ASA) at room temperature. The inset shows 5-ASA structure.

FIG. 22 is a graph showing the ATR-FTIR spectrum (at room temperature) of the control and the 5-ASA powder after 8 hours under vacuum at 200° C.

FIG. 23 shows a thermogravimetric analysis (TGA) of 5-ASA powders with and without ALD coating and their first derivative. The graphs indicate that the first derivative of the TGA curves (DTG) yields inflection points given as, 5-ASA (Control): 271.8° C., 300CyAl2O3@5-ASA: 267.9° C., and 200CyZnO@5-ASA: 269.6° C.

FIG. 24 is a graph showing an exemplary UV-vis spectra obtained from kinetic studies of 8 mg of 5-ASA in pH 4 media under no stir conditions. The increase in the 298 nm peak is shown as a function of time. For clarity, each spectrum is 250 seconds apart, though the actual data obtained was obtained with 5 second intervals.

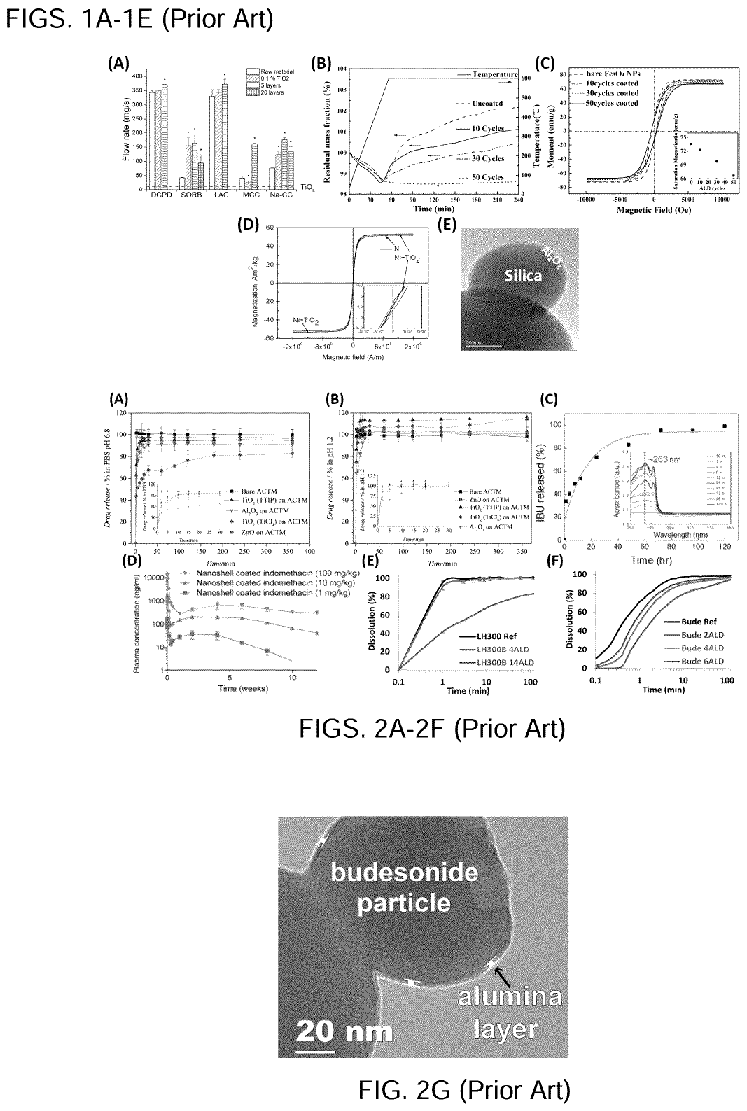

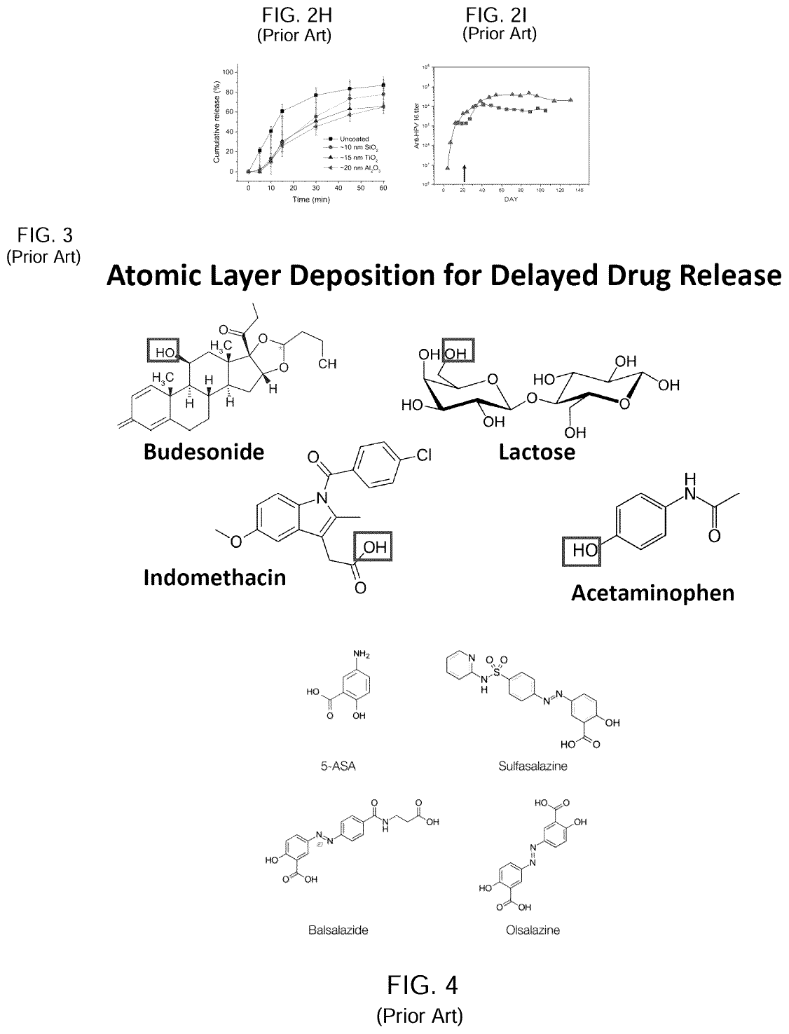

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described methods, active pharmaceutical ingredients, coated active pharmaceutical ingredients, drug coatings, metal oxide film coatings, treatment protocols, procedures, techniques, elements, and/or compositions along with any further application of the principles of the invention as described herein, are contemplated as would normally occur to one skilled in the art to which the invention relates.

### Introduction to Experimentation

The incidence of chronic illnesses worldwide continues to increase. To date, one out of three people are affected by multiple chronic illnesses globally¹¹. According to the World Health Organization, 71% of deaths reported worldwide every year are due to chronic illnesses. The biomedical and pharmaceutical industries have therefore prioritized research and development of new treatment alternatives for chronically ill patients. Increasing patient compliance and reducing the harsh side effects patients experience taking daily medication is at the core of developing delayed drug release systems by the pharmaceutical industry¹²⁻¹⁵. Achieving release over long periods of time (days to months) of active pharmaceutical materials has been accomplished through pellet polymeric coatings and matrices¹⁶. However, some challenges remain in terms of increasing loading capacity of the drug, having systems that mix the targeting and delaying ability of current drug release systems, and providing dosages that affect patients to the minimum^{17, 18}.

Aminosalicylates are a type of pharmaceutical molecule that have a common moiety in their molecular structure¹⁹. These molecules include sulfasalazine, olsalazine, balsalazide, and 5-Aminosalicylic (5-ASA) acid (FIG. 4). Aminosalicylates are first line therapy used for treating early stages of rheumatoid arthritis and inflammatory bowel disease.

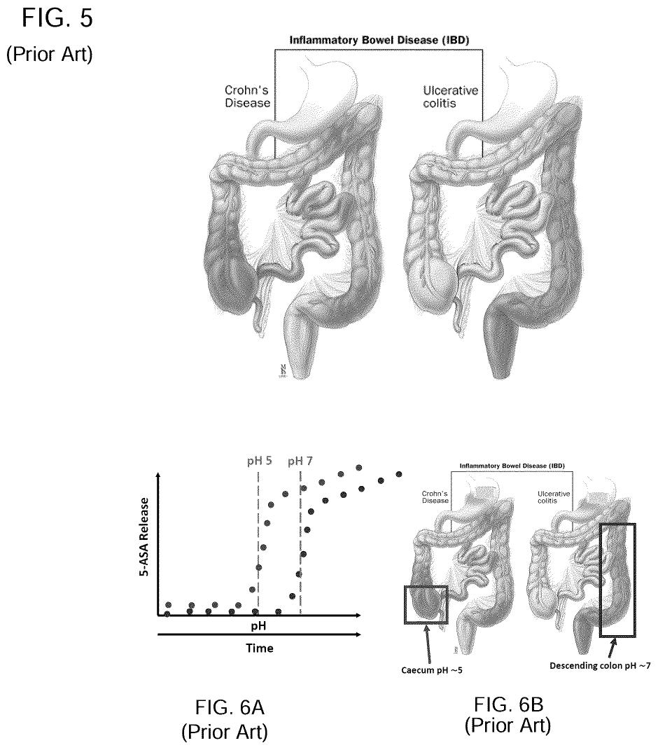

It can be seen in FIG. 4 that the moiety 5-ASA is present on either, or on both sides, of the azo-bond of these structures and is the therapeutic component for treating inflammation. The azo-bond is typically broken down by azo-reductase bacteria in the colon, and 5-ASA and the inactive metabolite are released¹⁹. The 5-ASA molecule scavenges reactive oxygen species (ROS) and blocks leukocyte activity and tumor necrosis factor α (TNF-α) responsible for chronic inflammation in both subtypes of inflammatory bowel disease (IBD): ulcerative colitis (UC) and Crohn's disease (CD). In the latter, the disease targets mostly the last section of the large intestine and the sigmoid colon, and it can continue to expand proximally towards the descending colon as shown in FIG. 5. UC only causes damage to the mucosal layer of the gastrointestinal (GI) tract wall and does not perforate through its different layers. The former, on the other hand, affects patients chronically, unpredictably, and with characteristic skip lesions anywhere throughout the GI wall (FIG. 5).

When 5-ASA is prescribed as an oral formulation, it is commonly absorbed in proximal structures of the small intestine. Therefore, when lesions in distal or even more proximal organs along the GI tract are affected, as in the case of CD, it is more difficult to achieve optimal 5-ASA absorption. As a result, delayed release systems and targeted release systems for 5-ASA have been extensively explored (FIGS. 6A-B).

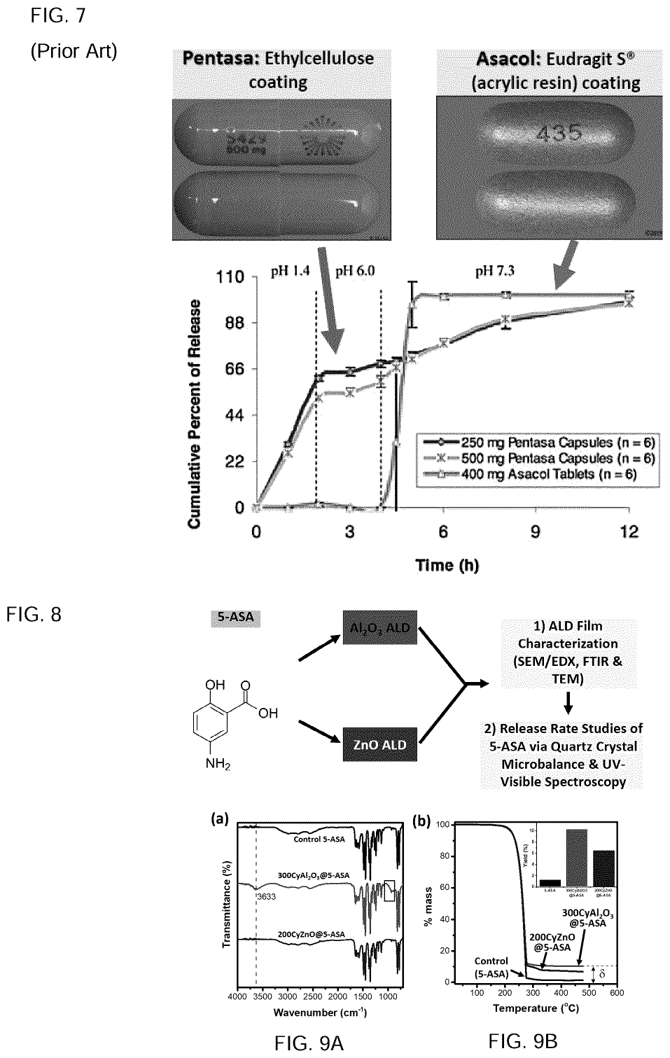

The drug release systems that are currently used for 5-ASA delivery are classified in delayed/extended release and targeted release systems. Delayed release systems deliver 5-ASA over long periods of time, and do not have control over the pH at which most of the 5-ASA is released. These systems include Pentasa® and Lialda®¹⁹, which achieve extended-release times through ethylcellulose coatings corresponding to a treatment time after oral administration of 5 days. Adult and pediatric patients experience long-term inflammation benefit from delayed release formulations since the low dosages enable patients to avoid taking daily medication, and the lower dosages prevent harsh side effects. However, due to the non-specific slow release of 5-ASA, if a patient experiences more severe lesions at colonic sites, the drug concentration once it arrives at the colon will be significantly diminished (FIG. 7).

The other type of release system is targeted release. These formulations use acrylic resin coatings and include Asacol®, Salofalk® and Claversal®¹⁹. In this case, the release of 5-ASA takes place mostly at basic pH, such as that of the colon and rectum (pH ˜7). Although beneficial to reach distal structures of the GI tract, no systems have been created to release most of 5-ASA at more acidic sites in a targeted manner and the treatment time of these systems is shorter and requires daily intake (FIG. 7).

To overcome the challenges of the drug release systems mentioned above, this invention proposes the synthesis of a single system in which both targeted and delayed release can be achieved. To this end, the instant inventors propose that using a coating technique called atomic layer deposition (ALD) as a method to create nanometer scale aluminum oxide (Al₂O₃) and zinc oxide (ZnO) coatings on 5-Aminosalicylic acid (5-ASA) powders enables control of 5-ASA release at specific pH profiles as well as over extended time periods.

### Experimentation

Release Rate Studies of 5-Aminosalicylic Acid (5-ASA) Coated with Atomic Layer Deposited Aluminum Oxide (Al₂O₃) and Zinc Oxide (ZnO) in an Acidic Environment

5-aminosalicylic acid (5-ASA) is a first line defense drug used to treat mild cases of inflammatory bowel disease. When administered orally, the active pharmaceutical ingredient is released throughout the gastrointestinal (GI) tract relieving chronic inflammation. However, delayed and targeted released systems for 5-ASA to achieve optimal dose volumes in acidic environments remains a challenge.

The experiments described herein demonstrate the application of atomic layer deposition (ALD) as a technique to synthesize nanoscale coatings on 5-ASA to control its release in acidic media. ALD Al₂O₃ (38.0 nm) and ZnO (24.7 nm) films were deposited on 1 g batch powders of 5-ASA in a rotatory thermal ALD system. Fourier transform infrared spectroscopy, scanning electron microscopy and scanning/transmission electron microscopy establish the interfacial chemistry and conformal nature of ALD coating over the 5-ASA particles. While Al₂O₃ forms a sharp interface with 5-ASA, ZnO appears to diffuse inside the 5-ASA. The release of 5-ASA is studied in pH 4 solution via UV-vis spectroscopy. Dynamic stirring, mimicking gut peristalsis, causes mechanical attrition of the Al₂O₃ coated particles, thereby releasing the 5-ASA. However, under static conditions lasting 5000 seconds, Al₂O₃ coated particles release only 17.5% 5-ASA compared to 100% release with ZnO coating. Quartz crystal microbalance based etch studies confirm the stability of Al₂O₃ in pH 4 media, where the ZnO films etch 41× faster than Al₂O₃. Such results are significant in achieving a nanoscale coating-based drug delivery system for 5-ASA with controlled release in acidic environments.

### Atomic Layer Deposition (ALD) Technology in Treatment of Chronic Illness

Chronic illnesses affect one out of three people globally¹. Within this class of diseases, inflammatory bowel disease (IBD) and its two sub-types, ulcerative colitis (UC) and Crohn's disease (CD), are classified as chronic illnesses associated with the human gastrointestinal (GI) tract which affects more than 6.8 million people, annually² (FIG. 5, FIG. 6B). Management and control of these illnesses is done through therapeutic intervention, where the primary goal is to increase patient compliance and reduce medication-based side effects. The primary pharmaceutical molecules to treat IBD are a class of aminosalicylates that have a common moiety: 5-aminosalicylic (5-ASA) acid in their molecular structure³ (FIG. 4). The 5-ASA scavenges reactive oxygen species (ROS), blocks leukocyte activity and tumor necrosis factor α (TNF-α) responsible for chronic inflammation⁴⁻⁶.

The key to effective therapeutic intervention is the delivery of the 5-ASA at the optimal dose-level over sustained periods of time or at a specific location along the GI tract (FIG. 6A). When 5-ASA is prescribed as an oral formulation, it is commonly absorbed in proximal structures of the small intestine. Therefore, when lesions in distal or even more proximal organs along the GI tract are affected, as in the case of CD, it is more difficult to achieve optimal 5-ASA absorption. As a result, delayed release systems and targeted release systems for 5-ASA have been extensively explored⁷⁻⁸.

Delayed release systems deliver 5-ASA over long periods of time⁹⁻¹² through ethylcellulose coatings corresponding to a treatment time after oral administration of approximately 5 days. These systems include Pentasa® and Lialda®³ (FIG. 7). However, due to the non-specific slow release of 5-ASA, drug release around severe lesions, especially at colonic sites, is significantly diminished. On the other hand, in targeted release systems, the delivery of 5-ASA takes place mostly at basic to neutral pH, such as that of the colon and rectum. This is achieved through enteric coatings such as, Eudragit S® that dissolve under basic pH in the GI tract¹³. These systems include Asacol®, Salofalk® and Claversal®³ (FIG. 7). Although beneficial to reach distal structures of the GI tract, no systems have been created to release most of 5-ASA at more acidic sites in a targeted manner. As a result, the treatment time of these systems is shorter and requires daily medicine intake.

Compounding these challenges is the fact that binder and enteric formulations used for coating active pharmaceutical ingredients (APIs) add volume to the pill, limiting API loading efficiencies. This hinders the development of high dosage oral formulations which are multifunctional i.e., pills that not only release 5-ASA over time but can release at specific locations along the entirety of the GI tract (1.5≤pH≤7.5) (FIG. 6A).

In this invention, the instant inventors propose the use of a nanoscale coating technology platform called atomic layer deposition (ALD) to coat 5-ASA powders to address the challenges described above/herein. ALD is a gas-phase, conformal, layer-by-layer film growth technique in which control over film thickness and composition can be achieved by sequential and alternative pulsing of gaseous precursor molecules¹⁴⁻¹⁸. The rationale to use ALD for coating 5-ASA is based on several factors. First, the use of ALD-based metal oxide chemistries such as Al₂O₃, ZnO and TiO₂ are well-established¹⁹⁻²¹. A key advantage being that ALD occurs at relatively low temperatures (≤150° C.) to maintain the API's viability. Second, the metal oxide candidates presented above are considered biocompatible and present no known toxicity²²⁻²⁴. Third, ALD provides non-line of sight and self-limiting deposition characteristics that result in highly conformal and pin-hole free films that act as diffusion barriers around particles²⁵. Fourth, the use of nanoscale films to coat API powders provides flexibility in terms of adding multifunctionality to pills without adding to its bulk and thus, can result in maintaining high dosage levels.

ALD was successfully used on various API powders to i) change the surface physicochemical characteristics and thereby improve powder flowability and processability²⁶, or ii) to achieve controllable release rates of the drug molecule of interest. Specifically, it was shown that by coating irregularly shaped pharmaceutical powders using ALD, loading efficiency and stability of solid drugs was vastly improved resulting in better control over delaying drug release times. Molecules previously used as model APIs are acetaminophen, indomethacin, budesonide and human papilloma virus vaccine powders containing HPV16 L1 capsomeres²⁷⁻³¹.

In the experiments described herein, aluminum oxide (Al₂O₃) and zinc oxide (ZnO) ALD films are used to coat 5-Aminosalicylic acid (5-ASA) particles with the goal of fine-tuning release of the 5-ASA throughout the human gastrointestinal tract. The motivation for using these two film chemistries is that in acidic environments, Al₂O₃ is relatively stable, while ZnO can be chemically etched. Thus, the use of these two chemistries provides a preliminary proof-of-concept for coating 5-ASA particles while understanding factors which influence 5-ASA release rates under various conditions encountered in the GI tract, including gut peristalsis (i.e., mechanical attrition) and acidic environment (low pH). Through detailed powder characterization, a basic understanding of the interaction chemistry of the ALD film with the underlying 5-ASA has been established. Studies of ALD Al₂O₃ coated 5-ASA under various stirring conditions mimicking gut peristalsis are conducted to highlight the importance of particle attrition on release rates. Comparison of release rates of ALD Al₂O₃ and ALD ZnO coated 5-ASA under acidic (pH=4) conditions are studied and quantified to highlight the importance of chemical interaction of the coatings with the gut microenvironment.

### Experimental Details

### Methodology

5-Aminosalicylic acid (5-ASA) particle powder is coated with aluminium oxide (Al₂O₃) or zinc oxide (ZnO) using atomic layer deposition (ALD) followed by characterization of the resulting ALD films (SEM/EDX, Fourier-transformed infrared (FTIR) microscopy, TEM) and release rate (of 5-ASA) studies (quartz crystal microbalance and UV-visible spectroscopy). A schematic illustration of this experimental methodology is shown in FIG. 8.

### Synthesis of Atomic Layer Deposition (ALD) Coated 5-Aminosalicylic Acid (5-ASA) Particle Powders

5-ASA particle powders (CAS 89-57-6, 95% purity) with a molecular weight of 153.14 g/mol were purchased from Sigma Aldrich®. The powder was batched into 1 g quantity of material for coating using ALD. A home-built viscous flow, rotatory thermal ALD reactor was used to coat 5-ASA powder via ALD and has been described in detail in Matthieu Chazot et al.³². Briefly, the ALD reactor consisted of a horizontal tube furnace (single zone Lindberg® Blue M) with a 7.62 cm core diameter and a length of 61 cm. The upstream side of this furnace was connected to a gas manifold with precursors attached via VCR® fittings to ALD Swagelok® valves. The valves were controlled via process recipes written with a Labview® control software. A digital mass flow controller (Parker® Porter Series II 601CV) was used to control the flow of argon (Airgas®, 99.999%) during the purge steps. A Leybold® Trivac vacuum pump (with Fomblin® oil) was used to pump the furnace reactor to a base pressure of ˜13.33 Pa. Pressure was monitored via an MKS® 910 DualTrans Transducer.

A rotatory barrel reactor was used to load 5-ASA powders inside the furnace ALD system. This system has been described in detail in Matthieu Chazot et al.²⁵. Briefly, the rotatory barrel reactor was made from stainless steel and measured 5.08 cm in length, 3.81 cm in outer diameter, and 2.79 cm in inner diameter. To avoid powders from agglomerating with each other or sticking to the inner walls of the reactor, a PTFE tube from McMaster-Carr®, was cut to fit the inside dimensions of the reactor. The powder sample was enclosed inside the barrel with a stainless-steel cap machined with 4 screw slots. In addition, the cap had openings for gases to flow in, through and out of the barrel reactor. The entire assembly was loaded into the furnace reactor from the load lock. A magnetically coupled arm (from Transfer Engineering and Manufacturing®) was used to slide the rotatory reactor inside the furnace. An external stepper motor was then used to rotate the rotatory reactor along its longitudinal axis inside the furnace through a magnetically coupled gear assembly at 8 rpm. The constant tumbling action ensured that the powder was in constant motion during the ALD process, thus exposing the entire batch of powder to the ALD precursors³³.

All ALD processes were conducted at 120° C. For aluminium oxide (Al₂O₃) coating, trimethyl aluminium (TMA, CAS 75-24-1, ≥97% purity, Sigma Aldrich®) and deionized (DI) water (Direct-Q Millipore®) were used. For zinc oxide (ZnO) coating, diethyl zinc (DEZ, CAS 557-20-0, ≥52 wt %, Sigma Aldrich®) and DI water were used. The recipe for the Al₂O₃ (ZnO) consisted of 1 second pulse of TMA (DEZ) and 0.5 second pulse of DI water. In between each pulse, the ALD furnace was purged with a 20 second flow of argon at 75 standard cubic centimetre per minute (sccm). This constituted one cycle of the ALD process. These steps were repeated until a specific number of cycles were met. After the process was completed, the rotatory barrel reactor was unloaded, and the powder collected and measured. The yield of the powder collected was ≥90% (≥0.9 g) after each run indicating minimal powder loss during the ALD process. Two standard recipes were run. One batch of 5-ASA was subjected to 300 cycles of Al₂O₃ ALD (300CyAl₂O₃@5-ASA); this corresponded to a thickness of 33.0 nm on a planar silicon substrate. Another batch of 5-ASA was subjected to 200 cycles of ZnO ALD (200CyZnO@5-ASA); this corresponded to a thickness of 30.6 nm on a planar silicon substrate. Details on the procedure for thickness measurement are provided below.

To measure ALD film thickness, separate coupons of silicon were run with the exact processes in stationary reactors and measured ex situ using a J. A. Woollam® M2000 spectroscopic ellipsometer with a wavelength range from 280 nm-1690 nm. All optical models for thin film analysis were built-in J. A. Woollam's Complete Ease software.

### Powder Characterization

Fourier-transformed infrared (FTIR) microscopy was performed on a Shimadzu AIM-9000 FTIR microscope. Attenuated total reflectance (ATR, Ge prism) FTIR was measured for each sample where the spectra were obtained by measuring a 100×100 μm spot size by 400 scans in a range from 700-4000 cm⁻¹, with a resolution of 4 cm⁻¹. The powders were placed on a 5×5 mm Si wafer (Ted Pella, Inc.) for measurements and a section of wafer without any powder was measured as the background. All spectra were baseline corrected, with CO₂ correction performed as necessary, within the AIM solution analysis software. Thermogravimetric analysis (TGA) of the powders was conducted on an ISI® TGA-1000 instrument, housed inside an inert nitrogen-atmosphere glovebox, using Pt sample pans and a 5 cm³/min flow of UHP N₂. The uncertainty in the mass measurement is +0.05 μg. The following protocol was conducted for all TGA experiments: 20-100° C. at a ramp rate of 20° C./min followed by 100-500° C. at a ramp rate of 5° C./min. The following mass of each sample was used: 2.14 mg 5-ASA, 2.16 mg of 300CyAl₂O₃@5-ASA, and 2.17 mg of 200CyZnO@5-ASA.

To characterize the morphology of 5-ASA powder before and after Al₂O₃ and ZnO ALD, a Hitachi® benchtop scanning electron microscope (SEM) with a 15 keV electron beam was used. A small amount of powder was placed on the SEM holder using double sided carbon tape. The powder was observed at an indicated magnification of 1200× using fast scan mode. Energy dispersive spectroscopy (EDS) was conducted using a Bruker Quantax EDX detector. The associated software from Bruker® was used for elemental identification of the 5-ASA powders, 300CyAl₂O₃@5-ASA and 200CyZnO@5-ASA. The x-ray counts were maximized by scanning large volumes of 5-ASA at multiple spots. At least three different regions on the samples were observed and measured.

Scanning/transmission electron microscopy was performed on the 300CyAl₂O₃@5-ASA particles using an FEI Tecnai® F30 S/TEM operated at 120 keV and equipped with a bottom-mounted Gatan® Multiscan 794 digital camera, Fischione Instruments Model 3000 high angle annular dark-field (HAADF) STEM detector, and an EDAX r-TEM super ultrathin window Si(Li) EDS system; both bright-field TEM (BF-TEM) and HAADF-STEM imaging were performing along with STEM-EDS mapping of Al, O, and C. TEM samples of 300CyAl₂O₃@5-ASA were prepared by making a 50:50=H₂O: isopropyl alcohol (IPA) solution in a 1 mL centrifuge tube and adding a small amount of 300CyAl₂O₃@5-ASA particles. 10 μL of the mixed solution was then dropped onto a 300-mesh Cu grid (63 μm grid openings) with a lacey C support film (SPI Supplies Inc.) and allowed to dry overnight.

Similarly, scanning/transmission electron microscopy was also performed on the 200CyZnO@5-ASA particles but using an FEI Themis Z S/TEM with Cs probe correction operated at 200 kV and equipped with a bottom-mounted FEI Ceta 16M CMOS camera, Fischione Instruments Model 3000 high angle annular dark-field HAADF-STEM detector, and a SuperX windowless Si drift detector EDS system (solid angle of collection=0.67 sr). Both high-resolution TEM (HR-TEM) and HAADF-STEM imaging were performed along with STEM-EDS mapping of Zn, O, and C. For HR-TEM imaging, the beam current was set to 300 pA, and a 60 μm objective aperture was used (resulting in ˜15 mrad semi-angle of collection). For HAADF-STEM imaging and STEM-EDS mapping, the probe semi-angle of convergence was 9 mrad with a probe current of ˜60 pA. It should be noted that the 5-ASA particles were susceptible to beam-induced damage during S/TEM analysis at 200 kV, often experiencing severe warping upon accumulation of a sufficiently high electron dose. Thus, the currents used for S/TEM analysis at 200 kV (as well as the analysis time used for STEM-EDS mapping) were selected to limit the effects of beam-induced damage, so the obtained data was accurately representative of the 200CyZnO@5-ASA particles. Unlike, the TEM samples of 300CyAl₂O₃@5-ASA particles, TEM samples of 200CyZnO@5-ASA particles were prepared using an entirely dry method; a small amount of 300CyAl₂O₃@5-ASA powder was directly placed onto a 300-mesh Cu grid (63 μm grid openings) with a lacey C support film (Ted Pella, #01895). The grid was then picked up using a pair of locking tweezers and shook very gently to disperse the particles over the grid; dry air was subsequently used to remove any macroscopically visual particle clusters from the grid. At least two different particles per sample were observed and measurements taken.

Quartz crystal microbalance studies of the etch rates of ALD Al₂O₃ and ZnO films were made using an SRS® QCM200 system. The AT-cut quartz crystals with patterned Cr—Au electrodes and a resonance frequency of 5 MHz (from SRS®) were used. The quartz crystals were 2.54 cm in diameter and were coated with 300 cycles and 200 cycles Al₂O₃ and ZnO ALD, respectively using the same precursors as described previously. The ALD on QCMs were performed using a Fiji Gen2 ALD commercial system from Veeco®. It is not expected that the film composition or properties are hardware dependent.

Post-ALD, the QCM probe was loaded with a coated crystal and the frequency and resistivity were zeroed prior to each run. The QCM was submerged into an 80 mL beaker with 80 mL deionized water and a stirring rod rotating at 150 rpm. After 3 minutes of collected baseline, 0.678 μL of HCl were pipetted into the solution to achieve a pH 4 acidic solution. The mass loss (μg/cm²) over time was extracted in real time until no significant mass loss could be measured on the QCM, thus indicating that the films were fully etched from the surface of the crystal.

### Release Rate Studies

UV-visual spectroscopy was used as the method of choice for identifying 5-ASA in low pH solutions. To conduct static tests, a Shimadzu ultraviolet-visible (UV-Vis) 1800® scanning spectrophotometer was used to identify 5-ASA's signature peak³⁴ at 298 nm. 10 ml quartz cuvettes with a 1 cm path length were filled with hydrochloric acid (HCl, ACS reagent 37% (w/w) from Millipore Sigma®) solution maintained at pH 4. After the UV-vis spectrophotometer was auto-zeroed and baselined, known amounts of 5-ASA powder (70 μg/ml) were dropped into the sample cuvette and scanned over a wavelength range from 190-1100 nm.

All release rate studies of 5-ASA were performed in triplicate using 8 mg powder batches. An Agilent Technologies Cary® 60 UV-Vis spectrophotometer, equipped with a 1 cm path length fiber optic probe (C Technologies Inc.), was used to monitor the 298 nm peak. The probe was inserted into 100 mL HCl solutions maintained at pH 4. This acidic media was made by adding 0.848 μL HCl to a 100 mL graduated cylinder and brought up to volume with DI water. The HCl solutions were placed on a magnetic stir plate, and the solution was stirred at various rotations per minute (rpm). For the experiments reported, three fixed rpm were used: 400, 23 and no stir (i.e., 0 rpm). Since the peak of interest for 5-ASA was found to be at 298 nm, the UV-vis spectrophotometer was set to collect 999 scans from a range of 200-400 nm, every 5 seconds.

### Results and Discussion

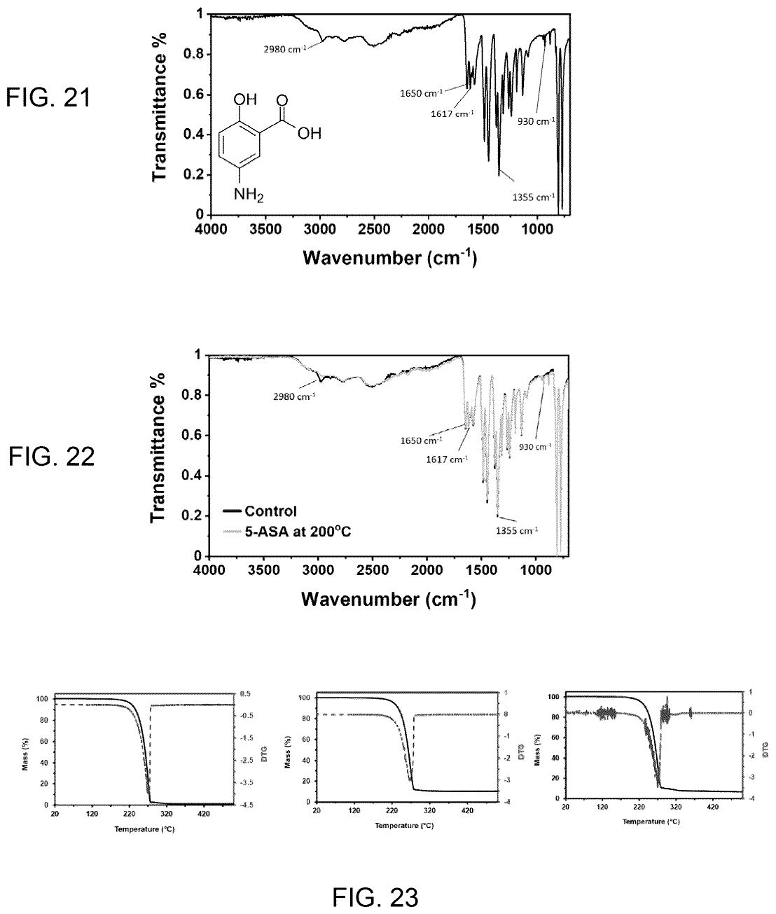

In order to establish viability of the 5-Aminosalicylic acid (5-ASA) post atomic layer deposition (ALD), the Fourier-transformed infrared (FTIR) spectra of the samples were studied first. The detailed FTIR spectrum of uncoated 5-ASA is well studied in literature³⁵⁻³⁸ and is also provided in FIG. 21 and Table A for the described powder along with the peak assignments table.

FIG. 21 is a graph showing the FTIR spectrum of 5-aminosalicyclic acid (5-ASA) control powder at room temperature. The inset shows 5-ASA structure. The 5-ASA (5-amino-2-hydroxybenzoic acid) molecule is composed of an aromatic benzene ring with a hydroxide (—OH) group in the “para” position to a primary amine (—NH2), and a carboxylic acid group in position 1 of the ring structure.

Five different peaks were identified as shown in FIG. 21 and listed in Table A. The peak at wavenumber 930 cm⁻¹ corresponds to an aromatic carboxylic acid vibration that has an out-of-plane deformation (G. Socrates). This peak is followed by a strong and sharp peak at 1355 cm⁻¹ indicating a C—N bond vibration at the primary amine site attached to the aromatic ring. This vibration is normally seen in primary and secondary aromatic amine groups. At 1617 cm⁻¹ there is a medium intensity peak corresponding to the bond between the primary amine nitrogen and hydrogen. The vibration type for this bond normally lies on the lower end of the wavenumber range for this FTIR peak. This is due to the amine group being part of a saturated aromatic system. In addition, the bending behavior of the N—H bonds is scissoring (G. Socrates). Adjacent to the N—H vibration peak, is a peak of equal intensity at 1650 cm⁻¹. This peak is assigned to intramolecular hydrogen bonded carboxylic acids. In the case of the 5-ASA molecule, the carbonyl oxygen of the carboxylic acid group ortho to the hydroxyl (OH) group is most likely hydrogen bonding leading to the formation of this peak (G. Socrates). Moreover, the peak at 2980 cm⁻¹ represents an ortho substituted OH group, which in this case is at a much lower intensity than that expected of an FTIR OH signal. The reduced intensity could be because of intramolecular hydrogen bonding with the adjacent carboxylic acid group on the 5-ASA molecule.

TABLE A

| Group | Peak (cm⁻¹) | Range (cm⁻¹) |
|---|---|---|
| N—H | 1620 | 1650-1580 |
| C—N | 1355 | 1300-1250 |
| C═O | 1650 | 1665-1610 |
| C═O | 1650 | 1680-1650 |
| ortho-OH | 2980 | 3200-2500 |
| COOH | 930 | 1000-900 |

Groups, peaks, and ranges of vibrations identified in 5-ASA FTIR.

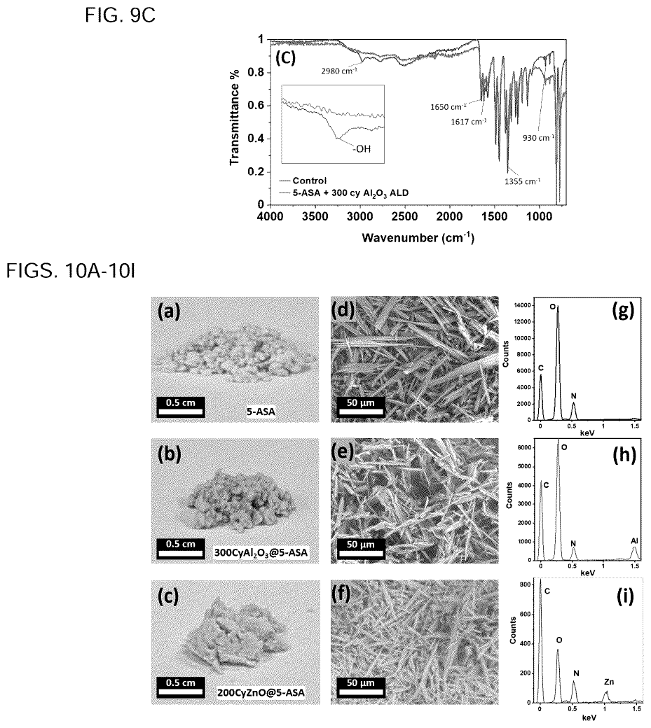

FIG. 9A shows a comparison between the ATR-FTIR spectra of the control (i.e., uncoated 5-ASA), 300CyAl₂O₃@5-ASA, and 200CyZnO@5-ASA. All ATR-FTIR spectra, which measures the bulk powder, contain characteristic 5-ASA peaks, including those at 1354 (C—N stretching), 1452 and 1487 (aromatic C—C stretching), 1620 (N—H bending), 1649 (C═O stretching), and a broad band from 2200-3200 cm⁻¹ (hydrogen bond vibrations) 35. The broad peak centered at 3633 cm⁻¹ in the 300 cycles ALD Al₂O₃ spectrum is due to O—H stretching and the broad peak from 850-1050 cm⁻¹ is attributed¹⁹ to amorphous Al₂O₃. Zn—O bands occur at wavenumbers below 700 cm⁻¹ and thus, are not seen in our spectra³⁹⁻⁴¹. As a further confirmation for the thermal stability of the 5-ASA powder FIG. 22 provides the ATR-FTIR spectra of the control and 5-ASA powder baked at 200° C. under vacuum (16 Pa). All peaks remain intact. This suggests that the 5-ASA powder is chemically stable to a temperature of at least 200° C. for 8 hours under vacuum.

An additional comparison between the ATR-FTIR spectra of the control (uncoated 5-ASA) and 300CyAl₂O₃@5-ASA is shown in FIG. 9C.

Thermogravimetric analysis (TGA) was conducted in inert N₂ atmosphere and is shown in FIG. 9B. The melting point for 5-ASA is given as 283° C. (from specification sheet, Sigma Aldrich®), while others⁴² have reported a melting point of 278° C. and a degradation temperature of 298° C. Thus, any change to the mass loss behavior would indicate degraded physical and chemical characteristics of 5-ASA. All three TGA curves resulted in approximately the same shape, with mass loss beginning ˜200° C. and ending ˜280° C. The ceramic yield ‘δ’ of the Control, 5-ASA was 1.32%. The 300CyAl₂O₃@5-ASA on 5-ASA ended with δ=10.30% and the 200CyZnO@5-ASA had a 8=6.50%. The inset in FIG. 9B, highlights these changes to δ. The greater ceramic yield of the ALD coated 5-ASA powders is consistent with thicker Al₂O₃ film on 5-ASA compared to the ZnO coated sample (discussed below). TGA data along with the first derivative of individual samples are shown in FIG. 23. The first derivative of the TGA curves resulted in inflection points at 271.8° C. for 5-ASA, 267.9° C. for 300CyAl₂O₃@5-ASA, and 269.6° C. for 200CyZnO@5-ASA showing minimal change in the powder characteristics upon ALD.

### Physical Morphology of the 5-Aminosalicylic Acid (5-ASA) Particles

In order to ascertain the physical morphology of the 5-ASA particles, optical images were taken for the control, 300CyAl₂O₃@5-ASA, and 200CyZnO@5-ASA powders. These are shown in FIGS. 10A-C, respectively. As received, 5-ASA powder is a light beige in color, whereas the 300CyAl₂O₃@5-ASA powder is darker. There is no change in color observed for 200CyZnO@5-ASA.

FIG. 10D shows the morphology of the 5-ASA powder at a magnification of 1200×. 5-ASA has a “needle” like crystalline structure 43-44 and a high degree of aggregation among the 5-ASA particles is observed. This behavior is likely due to intramolecular and van der Waals interactions between the particles and the hydroscopic nature of 5-ASA³³. The morphology of the particles remains intact through deposition of both 300 cycles Al₂O₃ ALD (FIG. 10E) and 200 cycles ZnO ALD (FIG. 10F).

FIG. 10G shows the EDS (energy dispersive) spectrum of control 5-ASA. High counts of carbon (0.277 keV), oxygen (0.392 keV), and nitrogen (0.525 keV)⁴⁵ are observed, consistent with the elements present in the 5-ASA molecule. 300CyAl₂O₃@5-ASA shows an additional Al peak at 1.486 keV (FIG. 10H) and suggests that the powders have been coated with Al₂O₃ ALD. Similarly, 200CyZnO@5-ASA (FIG. 10I) shows an energy peak corresponding to the presence of Zn (1.012 keV). Once again, the emission of x-rays corresponding to x-ray energies for Zn, suggests the presence of a ZnO ALD coating on 5-ASA.

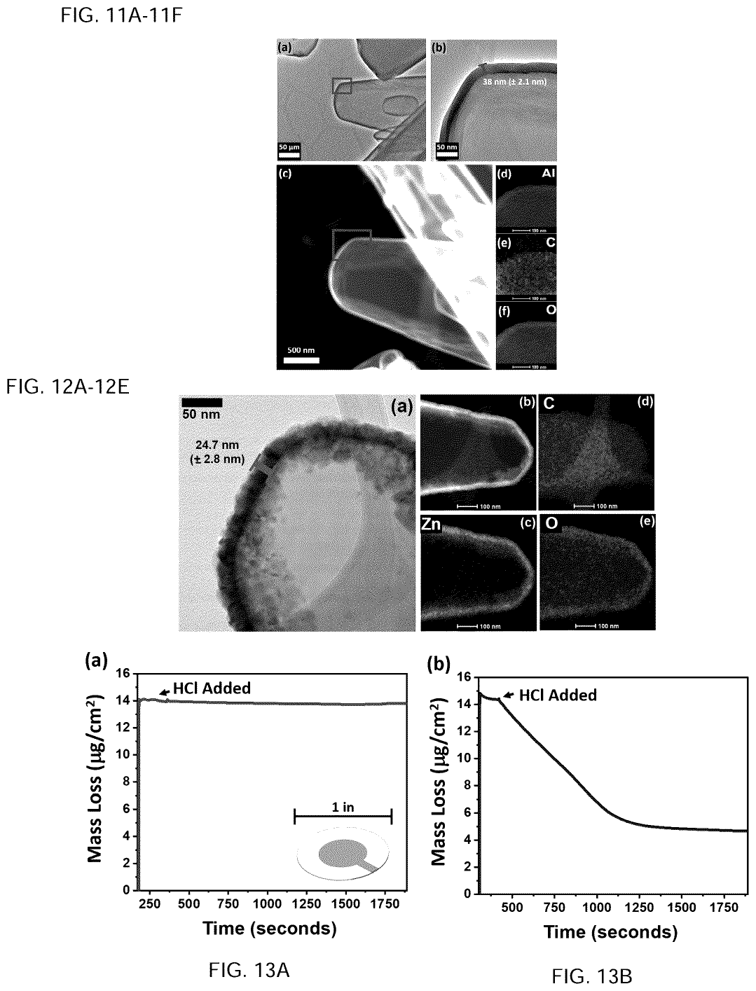

The 300CyAl₂O₃@5-ASA particles were analyzed under Bright-field transmission electron microscopy (BF-TEM). A typical cluster of 5-ASA particles are shown in FIG. 11A. Notice that the needle like morphology observed previously in SEM images are now visualized as large conical structures at higher magnification under the TEM. The ALD film can be observed as a dark conformal outline to the particles. This is typical of ALD coating behavior on particles and highlights the non-line-of-sight deposition characteristic of ALD³². To analyze the film thickness, FIG. 11B was captured at a higher magnification. At this magnification, the Al₂O₃ film thickness was measured using ImageJR software and the resulting thickness was found to be 38±2.1 nm around the perimeter of the coated 5-ASA particle. For a 300 cycle ALD process, this corresponds to a growth per cycle of 0.126 nm/cycle. The growth per cycle agrees with the typical growth per cycle for Al₂O₃ ALD, which is 0.11-0.13 nm/cycle⁴⁶.

The 300CyAl₂O₃@5-ASA particle was also imaged in dark field mode (FIG. 11C) to highlight the presence of the Al₂O₃ layer. The images, FIGS. 11A-C, show the conformational coating of ALD Al₂O₃ on the surface of 5-ASA particles. The ALD Al₂O₃ coats the 5-ASA particles like a blanket without pinholes or porosities. This provides an ideal barrier to dissolution of the 5-ASA in solutions of varying pH.

STEM-EDS mapping was performed and in FIG. 11D, Al elemental map is shown with a higher intensity for Al at the perimeter of the 5-ASA particle. This signifies the presence of elemental Al on the surface of the particle as a coating⁴⁷⁻⁴⁸. FIG. 11E shows the carbon signal with a background from the lacey carbon of the TEM copper grid. The primary carbon still appears to originate from 5-ASA. FIG. 11F shows the oxygen distribution with a higher concentration at the edge, signifying that, like Al, the O presence is stronger on the surface i.e., from the Al₂O₃ ALD film.

FIG. 12A shows the high-resolution transmission electron microscopy (HR-TEM) image of 200CyZnO@5-ASA particle. As in the case of the 300CyAl₂O₃@5-ASA, the ZnO film is also observed as a conformal layer around the 5-ASA. The thickness of the film is measured to be 24.7±2.8 nm. For a 200 cycles process, this corresponds to a growth per cycle of 0.12 nm/cycle and is lower than the usually reported growth per cycle of 0.17 nm/cycle⁴⁹. The film is polycrystalline in nature. This is to be expected as ALD ZnO, compared to ALD Al₂O₃, is deposited in its crystalline state even at low (≥ 100° C.) deposition temperatures⁵⁰. Finally, infiltration of the ZnO inside the 5-ASA particle was observed. The infiltration extends tens of nanometers inside the 5-ASA surface. This result is different from the Al₂O₃ coated sample which forms a clear, sharp interface with 5-ASA. High-angle annular dark field-scanning transmission electron microscopy (HAADF-STEM) imaging was also performed to further highlight the presence of the ZnO film, as shown in FIG. 12B. The ZnO crystals can be seen inside the 5-ASA in the contrast image as well. STEM-EDS mapping of Zn, C and O in the same area are shown in FIGS. 12C, D, and E, respectively. The Zn elemental map supports the observation that the ZnO penetrates the 5-ASA particle. It was hypothesized that the lower growth per cycle and the diffusion of the ZnO inside the 5-ASA are correlated. The initial pulses of DEZ could infiltrate the 5-ASA surface and cause growth of sub-surface ZnO. As a result, the final thickness obtained could be lower. This is not unreasonable as the largest interplanar spacing³⁶ for crystalline 5-ASA is 0.59 nm and the DEZ can be approximated as a one-dimensional molecule 0.52 nm long⁵¹. Taken together, the TEM results from 300CyAl₂O₃@5-ASA and 200CyZnO@5-ASA powders indicate that ALD process deposits conformal films around the 5-ASA particles. The thickness measurements are in line with TGA results (FIG. 9B) which show a ceramic yield (8) that is higher for 300CyAl₂O₃@5-ASA (38.0 nm) compared to 200CyZnO@5-ASA (24.7 nm).

To understand the etching rate of Al₂O₃ and ZnO ALD films, QCM crystals were coated by ALD. The inset on the lower right of FIG. 13A, shows a QCM crystal and its diameter corresponding to 1 inch (25.4 mm) and a gold electrode surface area of 1.267 cm². The crystal was coated with 300 cycles Al₂O₃ corresponding to a final thickness of 33 nm measured via ellipsometry on planar Si. After film deposition, the crystal was loaded onto a QCM holder, and was placed in 80 ml of DI water maintained at room temperature and with a rapidly rotating stir bar at the bottom of the beaker. The initial mass per cm² on the QCM crystal was around 14 μg/cm². After achieving a steady state in the resonance frequency (i.e., ˜5 MHZ) of the crystal (for approximately 4 minutes), 0.625 μL HCl was pipetted into the DI water to turn the solution acidic (corresponding to pH 4; 0.0001 M HCl). The low pH did not etch the Al₂O₃ coating due to this material's resistance to acidic media¹⁷. The slope of the curve corresponds to the etch rate of the film, and a slope of 0.000169 μg/cm²/s was extracted. In other words, 0.000169 μg of Al₂O₃ were etched away per cm² of the coated crystal per second. Given that the density⁵² of amorphous ALD Al₂O₃ is 3.1 g/cm³, the above etch rate corresponds to 0.0005 nm/s. This etch rate can be compared to those reported⁵³⁻⁵⁴ for untreated ALD Al₂O₃ where, for example, under strong acidic conditions (1 M H₂SO₄), 40 nm films can be removed in 2 days, yielding an etch rate of 0.0002 nm/s.

In contrast, a different QCM crystal was coated with 200 cycles ZnO ALD (FIG. 13B). The thickness measured via ellipsometry was 27 nm on planar Si. The starting mass per cm² for this film was around 14.6 μg/cm². ZnO can be etched away in acidic media as seen in previous reports⁵⁵. Therefore, the absolute value of the slope in this case is higher than that of the Al₂O₃, corresponding to an etch rate of 0.01259 μg/cm²/s. Given that the density⁵² of ALD ZnO is 5.61 g/cm³, the above etch rate corresponds to 0.02 nm/sec. Compared to Al₂O₃, ZnO etches 41× more rapidly than Al₂O₃.

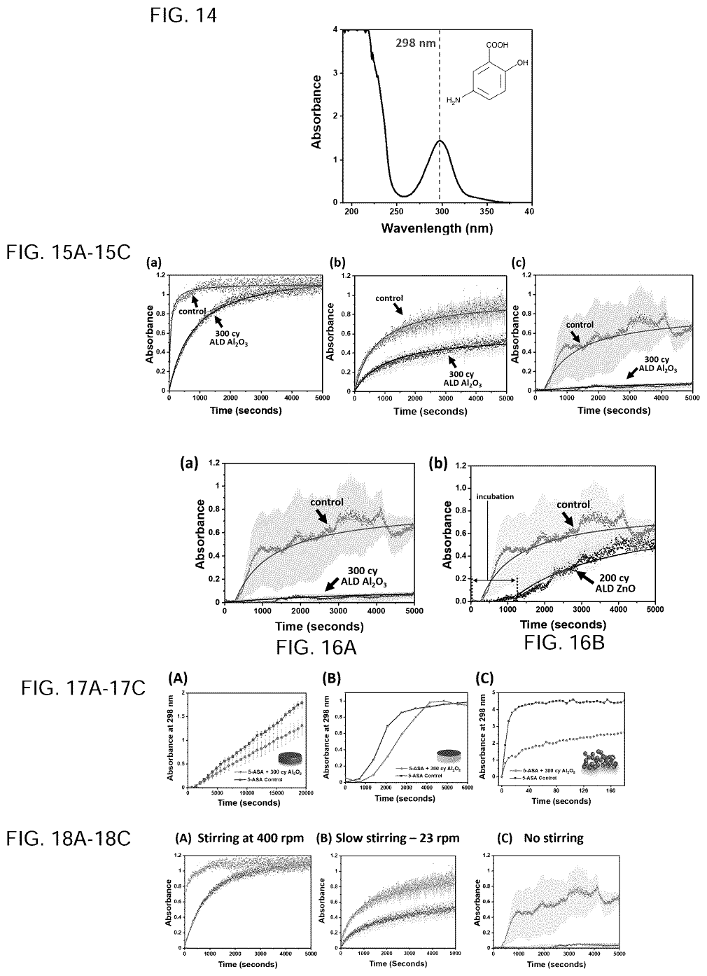

Prior to studying the release rate of 5-ASA in acidic HCl media, the UV-vis spectrum of 5-ASA was studied. An aliquot of 5-ASA corresponding to 70 μg/mL, was pipetted into a 1 cm path length quartz cuvette. The spectrum collected is shown in FIG. 14, where a peak maximum is reached at 298 nm as reported by others^{34, 56}. This characteristic peak is a good indicator that the 5-ASA remains chemically viable through ALD coating and therefore, is chosen to subsequently study the release rate of the 5-ASA powder in pH 4 solutions and with various coatings of ALD films.

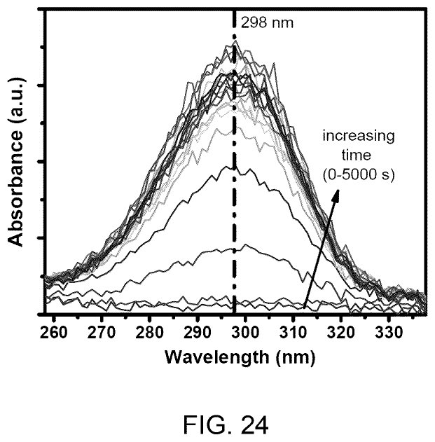

To study release rates of 5-ASA (uncoated and coated), powders were immersed in pH 4 HCl solution containing a fiber optic probe connected to a UV-vis system. The probe tip was consistently placed at the same position in the beaker to negate any artifact in the measurements which might arise from variabilities in the time taken for the molecule to reach the sensor tip. The release of 5-ASA in the solution was determined by tracking the evolution of 298 nm feature in the UV-vis spectrum over time. An example of the raw kinetic data collected is shown in FIG. 24 (UV-vis spectra obtained during release rate studies). FIG. 24 is a graph showing an exemplary UV-vis spectra obtained from kinetic studies of 8 mg of 5-ASA in pH 4 media under no stir conditions. The increase in the 298 nm peak is shown as a function of time. For clarity, each spectrum is 250 seconds apart, though the actual data obtained was obtained with 5 second intervals.

Next, two specific studies were conducted. In the first study, the release rate of 5-ASA powders, with and without 300 cycles Al₂O₃ ALD, was studied by gathering the absorbance at 298 nm as a function of time. The powders were stirred at different rpm using a magnetic stir bar in a pH 4 HCl solution. The justification for studying release rate as a function of stirring was to recognize that the stirring action produces mechanical attrition of the 5-ASA particles and may thus, mimic gut peristalsis. The powders were exposed to three different conditions: fast stirring at 400 rpm, slow stirring at 23 rpm, and no stirring (i.e., 0 rpm). The results for all three experiments are shown in FIGS. 15A, B, and C, respectively. The grey data points correspond to the mean of 3 batches (triplicates) of 8 mg each of control (i.e., uncoated 5-ASA) with standard deviations provided as a filled ‘band’ behind the data. The red data points correspond to 3 batches of 8 mg each of 300CyAl₂O₃@5-ASA. All time scans represent the absorption intensity of 5-ASA at 298 nm wavelength. In FIG. 15C, the standard deviation obtained for the no stir, uncoated sample was large as there was variation in the dispersity of the uncoated 5-ASA powder from batch to batch, where some particles sank to the bottom of the solution and the rest stayed afloat. The large standard deviation does not influence the conclusions obtained between uncoated and coated batches.

Across all stirring test conditions, the 300CyAl₂O₃@5-ASA powders showed subdued release compared to the control, uncoated powder. These results are in line with QCM data (FIGS. 13A-B) which shows enhanced chemical stability of Al₂O₃ in pH 4 solution. Thus, the release of 5-ASA in 300CyAl₂O₃@5-ASA can be attributed to particle attrition which leads to exposure of new, unprotected 5-ASA surfaces. The exposure of new 5-ASA surfaces lead to ready dissolution of the particles in pH 4 solution. This conclusion is further supported by the fact that the release rates for the coated samples are rpm dependent. At 400 rpm, nearly all 5-ASA coated powder is released in 5000 seconds due to the high rate of mechanical attrition. On the other hand, at 23 rpm while 5-ASA is released, the absorption value at the end of 5000 seconds is lower than that of the control powder. This indicates that the slow rpm is unable to break down all particles and these remain protected by the 300 cycle Al₂O₃ ALD shell. Finally, for the no stir (i.e., 0 rpm) sample, virtually no signal for 5-ASA is obtained indicating that the particles remain intact in solution, protected by the 300 cycle Al₂O₃ ALD shell over a sustained period of time. Thus, the results from the stirring experiment reiterate the fact that particle attrition effects in the gut due to peristalsis need to be taken into account when designing nano coatings for API powders.

In order to quantify release rate data, the following equation (Equation 1) was adopted to model the curves:

$$abs ( t ) = \frac{{P}_{1} t}{{P}_{2} + t} \qquad [ 1 ]$$

Here, abs (t) represents the time (1) variation of the absorbance from the UV-vis data. P₁ represents the asymptotic value in absorbance which corresponds to the full dissolution of 5-ASA in HCl. P₂ is the time taken in seconds for reaching half the absorbance saturation (i.e., the time at which absorbance is 0.5×P₁). In FIGS. 15A-C, the model curves are also overlaid over the raw data points as a solid line.

TABLE 1
Parameters P₁ and P₂ extracted for the release rate data from FIGS. 15A-
C. The P₁ is given in terms of absorbance (a.u.) and P₂ is provided in terms of seconds.
The definitions for P₁ and P₂ are provided in the text.

| | P₁ (absorbance, a.u.) | | | P₂ (time, seconds) | | |
|---|---|---|---|---|---|---|
| Coating | 400 rpm | 23 rpm | No rpm | 400 rpm | 23 rpm | No rpm |
| Control | 1.084 ± 0.002 | 0.961 ± 0.005 | 0.818 ± 0.007 | 41 ± 1 | 712 ± 8 | 1023 ± 31 |
| Al₂O₃ | 1.258 ± 0.003 | 0.595 ± 0.004 | 0.140 ± 0.008 | 784 ± 8 | 1070 ± 16 | 2686 ± 261 |

A first order kinetics equation is regularly used to describe release rates⁵⁷. In the instant case, the use of the Equation 1 justified for the following reasons. First, our first order kinetics modeling did not yield a high goodness of fit with the data; possibly attributed to a multi mechanism for release which involves i) mechanical action involving particle breakage exposing

fresh surfaces and, ii) slow shell dissolution in the low pH solution. Second, Equation 1 provides a high goodness of fit (r²>0.90, except for control sample in the no stir case) for all the release rate data evaluated. Third, the parameters (P) and P₂) extracted are physically relevant. P₁ corresponds to the saturation absorbance for a specific mass loading of the 5-ASA powder. P₂ is related to the kinetics of the dissolution process which directly corresponds to the release rate of the powder. Importantly, these parameters allow quantification of the ALD coating performance on 5-ASA which is key to comparing samples across various testing conditions and coating chemistries. The parameters extracted from fitting Equation 1 to the release rate data (average and standard deviation obtained from triplicate runs) for all powders are provided in Table 1. P₁ is found to be a function of both rpm and coating. P/increases with rpm and, in general, is higher for the control, uncoated powder compared to 300CyAl₂O₃@5-ASA. At 23 rpm P₁ is 0.961±0.005 for the uncoated powder and 0.595±0.004 for 300CyAl₂O₃@5-ASA. Similarly, for the no stir sample, P₁ is 0.818±0.007 for the uncoated powder and 0.140±0.008 for 300CyAl₂O₃@5-ASA. An exception to this is found for the 400 rpm sample where P₁ for the 300CyAl₂O₃@5-ASA powder appears higher (1.258±0.003) than the uncoated powder (1.084±0.002). This anomaly could be attributed to the difference in dispersity of the two powders, accentuated at high rpm.

The kinetic parameter P₂ quantifies the rate of dissolution of the 5-ASA in pH 4 solution. As expected, P₂ increases with decreasing stirring rate and is consistently higher for the coated powder than for the uncoated powder, indicating the effectiveness of the 300 cycles Al₂O₃ ALD in slowing dissolution across all stirring conditions. For example, at 400 rpm the uncoated powder has a P₂ of 41±1 seconds, whereas the 300CyAl₂O₃@5-ASA has a P₂ of 784±8 seconds. At 23 rpm, the uncoated powder has a P₂ of 712±8 seconds, whereas the 300CyAl₂O₃@5-ASA has a P₂ of 1070±16 seconds. Finally, for the no stir sample the uncoated powder has a P₂ of 1023±31 seconds, whereas 300CyAl₂O₃@5-ASA has a P₂ of 2686±261 seconds.

Next, the effect of two different film chemistries: 300 cycles Al₂O₃ ALD and 200 cycles ZnO ALD, were studied on the release rate of 5-ASA in pH 4 solution. The release rate studies were conducted under no stir conditions to negate any influence of mechanical attrition of the particles. The results are shown in FIG. 16A for 300CyAl₂O₃@5-ASA (same as FIG. 15C) and FIG. 16B for 200CyZnO@5-ASA.

As reported previously, 300CyAl₂O₃@5-ASA does not show significant release of 5-ASA as the Al₂O₃ is stable in pH 4. However, for 200CyZnO@5-ASA, an incubation period of 1225 seconds is observed. Beyond this time, 5-ASA can be reliably detected in the solution. The incubation time is the time it takes for HCl to etch through the ZnO coating. From QCM studies presented earlier, the etching rate for ZnO at pH 4 is 0.02 nm/s. Therefore in 1225 seconds, it is expected that 24.5 nm of ZnO film will be removed. This value can be compared to the 24.7 nm final thickness of the ZnO observed on 5-ASA shown in the TEM image in FIG. 12A. Additionally, no obvious effect of the infiltrated ZnO crystals on the release rates was seen. It is also possible that during deposition some of the 5-ASA particles get deposited with thickness lesser than the target thickness (e.g., new particles which form during the tumbling process in the rotatory ALD reactor). The incubation period observed for 200CyZnO@5-ASA is an indication that chemical interaction between the solution and the ZnO coating (i.e., its composition and thickness) can be used to drive dissolution based on 1) specificity of environment (i.e., pH) and 2) time.

The fit parameters P; and P₂ are presented as a comparison in Table 2. The P/parameter for the control and 200CyZnO@5-ASA are nearly the same indicating that, eventually with the ZnO fully dissolved, the 5-ASA powder reaches its saturation absorption value of ˜0.82, as in the case of the control. In the same time interval, 300CyAl₂O₃@5-ASA releases 5-ASA with an absorbance value of 0.140. This value is 17.5% of the total absorbance recorded from the control sample. Thus, the P₁ parameter helps quantify the degree of protection that Al₂O₃ provides in a pH 4 environment.

TABLE 2
Parameters P₁ and P₂ extracted for the
release rate data from FIGS. 16A-B.

| | P₁ (absorbance, a.u.) | P₂ (time, seconds) |
|---|---|---|
| Coating | No rpm | No rpm |
| Control | 0.818 ± 0.007 | 1023 ± 31 |
| Al₂O₃ | 0.140 ± 0.008 | 2686 ± 261 |
| ZnO | 0.820 ± 0.023 | 2651 ± 135 |

The kinetic parameter P₂, on the other hand, is similar for 300CyAl₂O₃@5-ASA and 200CyZnO@5-ASA (2686 seconds vs. 2651 seconds, respectively). Since in both cases the powder sinks to the bottom of the beaker (note: for the control, uncoated sample the powder is dispersed at the bottom and top and hence, results in lower P₂), the times involved in reaching saturation are diffusion limited and unrelated to the powder coating quality.

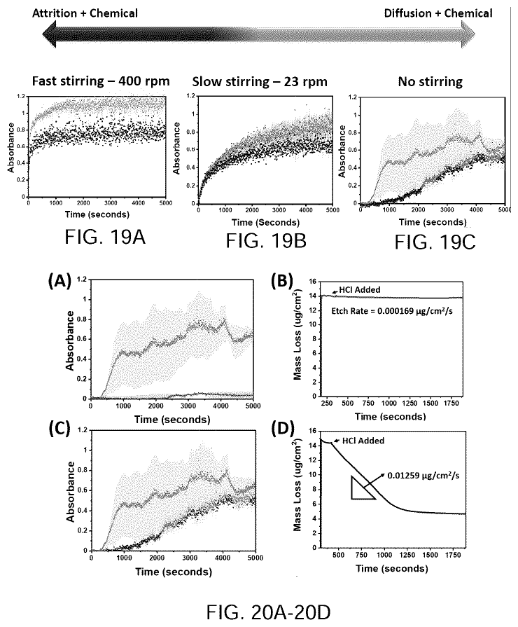

Additional data and comparisons regarding release rate studies are shown in FIGS. 17A-C, FIGS. 18A-C, FIGS. 19A-C, and FIGS. 20A-D.

Conformal nanoscale coatings of 38.0 nm of Al₂O₃ and 24.7 nm of ZnO on 5-ASA powders have been demonstrated using ALD. ATR-FTIR measurements and TGA analyses on the coated powders indicate that the low temperature of ALD (120° C.) maintains the viability of the molecule. SEM reveals a needle-like morphology of the 5-ASA that remains intact through the deposition process. EDS provides confirmation of the presence of Al₂O₃ and ZnO. S/TEM and associated STEM/EDS mapping of the powders indicate that ALD is uniform, pin-hole free and conformal around the 5-ASA particles. Furthermore, the interface between Al₂O₃ and 5-ASA is found to be stable and sharp whereas ZnO appears to diffuse limitedly inside the 5-ASA particle.

To study 5-ASA release rates in acidic environment, the etching rates of Al₂O₃ and ZnO at pH 4 were studied using a QCM. It was found that the etching rate of ZnO is 41× more than Al₂O₃. Based on this finding, 5-ASA powder with and without ALD Al₂O₃ and ZnO layers were tested in acidic, pH 4 media. The effect of stirring, mimicking gut peristalsis, was monitored for Al₂O₃ coated 5-ASA powders. High stirring releases the 5-ASA faster in the solution due to high particle attrition and exposure of new surfaces to the acidic solution. In contrast, the no stir ALD Al₂O₃ coated sample effectively slows the release of 5-ASA for at least 5000 seconds in pH 4 solution. The ALD ZnO coated 5-ASA sample under no stir conditions shows an initial ‘incubation period’ in which 5-ASA is prevented from being released. Once the ZnO is etched, 5-ASA readily dissolves in the pH 4 solution. Quantification of the release rate data indicates that the ALD Al₂O₃ coated sample releases only 17.5% of the total load in the time it takes to release the full load of 5-ASA for the ALD ZnO coated sample. The fundamental mechanistic studies reported herein show that ALD-based nanoscale coatings can be designed to create effective barriers around 5-ASA particles. The coatings can be optimally engineered to controllably release the molecule in low pH environments thereby improving therapeutic efficacy of controllable release drug systems.

### CONCLUSION

All patents and publications mentioned in this specification are indicative of the levels of those skilled in the art to which the invention pertains. All patents and publications are herein incorporated by reference to the same extent as if each individual publication were specifically and individually indicated to be incorporated by reference. It is to be understood that while a certain form of the invention is illustrated, it is not intended to be limited to the specific form or arrangement herein described and shown.

It will be apparent to those skilled in the art that various changes may be made without departing from the scope of the invention and the invention is not to be considered limited to what is shown and described in the specification. One skilled in the art will readily appreciate that the present invention is adapted to carry out the objectives and obtain the ends and advantages mentioned, as well as those inherent therein.

The methods, active pharmaceutical ingredients, coated active pharmaceutical ingredients, drug coatings, metal oxide film coatings, treatment protocols, procedures, techniques, elements, and/or compositions described herein are presently representative of the preferred embodiments, are intended to be exemplary and are not intended as limitations on the scope. Changes therein and other uses will occur to those skilled in the art which are encompassed within the spirit of the invention.

Although the invention has been described in connection with specific, preferred embodiments, it should be understood that the invention as ultimately claimed should not be unduly limited to such specific embodiments. Indeed various modifications of the described modes for carrying out the invention, which are obvious to those skilled in the art, are intended to be within the scope of the invention.

### REFERENCES

### Reference List A (Background, Brief Description of the Drawings, Introduction to Experimentation)

1. Skoog, S. A.; Elam, J. W.; Narayan, R. J., Atomic layer deposition: medical and biological applications. International Materials Reviews 2013, 58 (2), 113-129.
2. Hirschberg, C.; Jensen, N. S.; Boetker, J.; Madsen, A. Ø.; Kääriäinen, T. O.; Kääriäinen, M.-L.; Hoppu, P.; George, S. M.; Murtomaa, M.; Sun, C. C.; Risbo, J.; Rantanen, J., Improving Powder Characteristics by Surface Modification Using Atomic Layer Deposition. Organic Process Research & Development 2019, 23 (11), 2362-2368.
3. Duan, C.-L.; Deng. Z.; Cao, K.; Yin, H.-F.; Shan, B.; Chen, R., Surface passivation of Fe₃O₄ nanoparticles with Al₂O₃ via atomic layer deposition in a rotating fluidized bed reactor. Journal of Vacuum Science & Technology A: Vacuum, Surfaces, and Films 2016, 34 (4), 04C103.
4. Hakim, L. F.; Blackson, J.; George, S. M.; Weimer, A. W., Nanocoating Individual Silica Nanoparticles by Atomic Layer Deposition in a Fluidized Bed Reactor. Chemical Vapor Deposition 2005, 11 (10), 420-425.
5. Käariainen, T. O.; Kemell, M.; Vehkamaki, M.; Käariäinen, M.-L.; Correia, A.; Santos, H. A.; Bimbo, L. M.; Hirvonen, J.; Hoppu. P.; George, S. M.; Cameron, D. C.; Ritala, M.; Leskelä, M., Surface modification of acetaminophen particles by atomic layer deposition. International Journal of Pharmaceutics 2017, 525 (1), 160-174.
6. Seong. S.; Park. I.-S.; Jung, Y. C.; Lee, T.; Kim, S. Y.; Lee, S.-J.; Ahn, J., Fe₃O₄—ZnO Core-Shell Nanoparticles Fabricated by Ultra-Thin Atomic Layer Deposition Technique as a Drug Delivery Vehicle. Electronic Materials Letters 2019, 15 (4), 493-499.
7. Hellrup, J.; Rooth, M.; Mårtensson, E.; Sigfridsson, K.; Johansson, A., Nanoshells prepared by atomic layer deposition-long acting depots of indomethacin. European Journal of Pharmaceutics and Biopharmaceutics 2019, 140, 60-66.
8. Zhang. D.; Quayle, M. J.; Petersson, G.; Van Ommen, J. R.; Folestad, S., Atomic scale surface engineering of micro- to nano-sized pharmaceutical particles for drug delivery applications. Nanoscale 2017, 9 (32), 11410-11417.
9. La Zara, D.; Sun, F.; Zhang, F.; Franek, F.; Balogh Sivars, K.; Horndahl, J.; Bates, S.; Brännström, M.; Ewing, P.; Quayle, M. J.; Petersson, G.; Folestad, S.; Van Ommen, J. R., Controlled Pulmonary Delivery of Carrier-Free Budesonide Dry Powder by Atomic Layer Deposition. ACS Nano 2021, 15 (4), 6684-6698.
10. Garcea, R. L.; Meinerz, N. M.; Dong, M.; Funke, H.; Ghazvini, S.; Randolph, T. W., Single-administration, thermostable human papillomavirus vaccines prepared with atomic layer deposition technology. npj Vaccines 2020, 5 (1).
11. Hajat, C.; Stein, E., The global burden of multiple chronic conditions: A narrative review. Preventive Medicine Reports 2018, 12, 284-293.
12. Anderson, R. J.; Kirk, L. M., Methods of improving patient compliance in chronic disease states. Archives of Internal Medicine 1982, 142 (9), 1673-1675.
13. Eraker, S. A.; Kirscht, J. P.; Becker, M. H., Understanding and improving patient compliance. Annals of internal medicine 1984, 100 (2), 258-268.
14. Dunbar-Jacob, J.; Mortimer-Stephens, M., Treatment adherence in chronic disease. Journal of clinical epidemiology 2001, 54 (12), S57-S60.
15. Shale, M.; Riley, S., Studies of compliance with delayed-release mesalazine therapy in patients with inflammatory bowel disease. Alimentary pharmacology & therapeutics 2003, 18 (2), 191-198.
16. Trenfield, S. J.; Basit, A. W., Modified drug release: Current strategies and novel technologies for oral drug delivery. Elsevier: 2020; pp 177-197.
17. Li, X.; Lu, C.; Yang, Y.; Yu, C.; Rao, Y., Site-specific targeted drug delivery systems for the treatment of inflammatory bowel disease. Biomedicine & Pharmacotherapy 2020, 129, 110486.
18. Lautenschläger, C.; Schmidt, C.; Fischer, D.; Stallmach, A., Drug delivery strategies in the therapy of inflammatory bowel disease. Advanced drug delivery reviews 2014, 71, 58-76.
19. Campregher, C.; Gasche, C., Aminosalicylates. Best Pract. Res. Clin. Gastroenterol. 2011, 25 (4-5), 535-546.

### Reference List B (Detailed Description of the Invention-After Introduction to Experimentation)

(1) Hajat, C.; Stein, E. The global burden of multiple chronic conditions: A narrative review. Prev. Med. Rep. 2018, 12, 284-293, DOI: 10.1016/j.pmedr.2018.10.008.
(2) Alatab, S.; Sepanlou, S. G.; Ikuta, K.; Vahedi, H.; Bisignano, C.; Safiri, S.; Sadeghi, A.; Nixon, M. R.; Abdoli, A.; Abolhassani, H. The global, regional, and national burden of inflammatory bowel disease in 195 countries and territories, 1990-2017: a systematic analysis for the Global Burden of Disease Study 2017. Lancet Gastroenterol. Hepatol. 2020, 5 (1), 17-30.
(3) Campregher, C.; Gasche, C. Aminosalicylates. Best Pract. Res. Clin. Gastroenterol. 2011, 25 (4-5), 535-546, DOI: 10.1016/j.bpg.2011.10.013.
(4) Ahnfelt-Rønne, I.; Nielsen, O. H.; Christensen, A.; Langholz, E.; Binder, V.; Riis, P. Clinical evidence supporting the radical scavenger mechanism of 5-aminosalicylic acid. Gastroenterology 1990, 98 (5), 1162-1169.
(5) MacDermott, R. P. Progress in understanding the mechanisms of action of 5-aminosalicylic acid. Am. J. Gastroenterol. 2000, 95 (12). 3343.
(6) Peskar, B.; Dreyling, K.; May, B.; Schaarschmidt, K.; Goebell, H. Possible mode of action of 5-aminosalicylic acid. Dig. Dis. Sci. 1987, 32 (12), S51-S56.
(7) Chuong. M. C.; Christensen, J. M.; Ayres, J. W. New Dissolution Method for Mesalamine Tablets and Capsules. Dissolution Technol. 2008, 15 (3), 7-14, DOI: 10.14227/dt150308p7.
(8) Qiu, Y.; Lee, P. I. Rational Design of Oral Modified-Release Drug Delivery Systems. Elsevier: 2017; pp 519-554.
(9) Anderson, R. J.; Kirk, L. M. Methods of improving patient compliance in chronic disease states. Arch. Intern. Med. 1982, 142 (9), 1673-1675.
(10) Eraker, S. A.; Kirscht, J. P.; Becker, M. H. Understanding and improving patient compliance. Ann. Intern. Med. 1984, 100 (2), 258-268.
(11) Dunbar-Jacob, J.; Mortimer-Stephens, M. Treatment adherence in chronic disease. J. Clin. Epidemiol. 2001, 54 (12), S57-S60.
(12) Shale, M.; Riley, S. Studies of compliance with delayed-release mesalazine therapy in patients with inflammatory bowel disease. Aliment. Pharmacol. Ther. 2003, 18 (2), 191-198.
(13) Thakral, S.; Thakral, N. K.; Majumdar, D. K. Eudragit®: a technology evaluation. Expert Opin. Drug Discovery 2013, 10 (1), 131-149.
(14) George, S. M. Atomic layer deposition: an overview. Chem. Rev. 2010, 110 (1), 111-131.
(15) Johnson, R. W.; Hultqvist, A.; Bent, S. F. A brief review of atomic layer deposition: from fundamentals to applications. Mater. Today 2014, 17 (5), 236-246.
(16) Leskelä, M.; Ritala, M. Atomic layer deposition (ALD): From precursors to thin film structures. Thin Solid Films 2002, 409 (1), 138-146.
(17) Puurunen, R. L. Surface chemistry of atomic layer deposition: A case study for the trimethylaluminum/water process. J. Appl. Phys. 2005, 97 (12), 9.
(18) Miikkulainen, V.; Leskelä, M.; Ritala, M.; Puurunen, R. L. Crystallinity of inorganic films grown by atomic layer deposition: Overview and general trends. J. Appl. Phys. 2013, 113 (2), 2.
(19) Dillon, A. C.; Ott, A. W.; Way, J. D.; George, S. M. Surface-Chemistry of Al₂O₃ Deposition Using Al(CH₃)₃ and H₂O in a Binary Reaction Sequence. Surf. Sci. 1995, 322 (1-3), 230-242.
(20) Yousfi, E. B.; Fouache, J.; Lincot, D. Study of atomic layer epitaxy of zinc oxide by in-situ quartz crystal microgravimetry. Appl. Surf. Sci. 2000, 153 (4), 223-234, DOI: 10.1016/s0169-4332 (99) 00330-x.
(21) Sammelselg. V.; Rosental, A.; Tarre, A.; Niinisto, L.; Heiskanen, K.; Ilmonen, K.; Johansson, L. S.; Uustare. T. TiO₂ thin films by atomic layer deposition: A case of uneven growth at low temperature. Appl. Surf. Sci. 1998, 134 (1-4), 78-86, DOI: 10.1016/s0169-4332 (98) 00224-4.
(22) Dias. V.; Maciel. H.; Fraga, M.; Lobo, A. O.; Pessoa. R.; Marciano, F. R. Atomic Layer Deposited TiO₂ and Al₂O₃ Thin Films as Coatings for Aluminum Food Packaging Application. Materials 2019, 12 (4). DOI: 10.3390/ma12040682.
(23) Petrochenko, P. E.; Kumar, G.; Fu. W.; Zhang. Q.; Zheng. J.; Liang. C.; Goering. P. L.; Narayan. R. J. Nanoporous Aluminum Oxide Membranes Coated with Atomic Layer Deposition-Grown Titanium Dioxide for Biomedical Applications: An In Vitro Evaluation. J. Biomed. Nanotechnol. 2015, 11 (12). 2275-2285. DOI: 10.1166/jbn.2015.2169.
(24) Osorio. D.; Lopez. J.; Tiznado, H.; Farias, M. H.; Hernandez-Landaverde. M. A.; Ramirez-Cardona. M.; Yañcz-Limon, J. M.; Gutierrez. J. O.; Caicedo, J. C.; Zambrano, G. Structure and Surface Morphology Effect on the Cytotoxicity of [Al₂O₃/ZnO] Nanolaminates on/316L SS Growth by Atomic Layer Deposition (ALD). Crystals 2020, 10 (7), DOI: 10.3390/cryst10070620.
(25) Chazot. M.; Kostogiannes, A.; Julian, M.; Feit. C.; Sosa, J.; Kang. M.; Blanco, C.; Cook. J.; Rodriguez. V.; Adamietz, F.; Verreault. D.; Banerjee, P.; Schepler. K.; Richardson, M. C.; Richardson, K. A. Enhancement of ZnSe stability during optical composite processing via atomic layer deposition. J. Non-Cryst. Solids 2021. 121259. DOI: https://doi.org/10.1016/j.jnoncrysol.2021.121259.
(26) Hirschberg. C.; Jensen. N. S.; Boetker, J.; Madsen. A. Ø.; Kääriäinen. T. O.; Kääriäinen, M.-L.; Hoppu. P.; George. S. M.; Murtomaa. M.; Sun, C. C.; Risbo, J.; Rantanen. J. Improving Powder Characteristics by Surface Modification Using Atomic Layer Deposition. Org. Process Res. Dev. 2019, 23 (11). 2362-2368. DOI: 10.1021/acs.oprd.9b00247.
(27) Kääriäinen. T. O.; Kemell. M.; Vchkamaki. M.; Kääriäinen, M.-L.; Correia. A.; Santos, H. A.; Bimbo. L. M.; Hirvonen, J.; Hoppu. P.; George. S. M.; Cameron, D. C.; Ritala, M.; Leskelä. M. Surface modification of acetaminophen particles by atomic layer deposition. Int. J. Pharm. 2017, 525 (1). 160-174. DOI: 10.1016/j.ijpharm.2017.04.031.
(28) Hellrup. J.; Rooth. M.; Mårtensson, E.; Sigfridsson, K.; Johansson. A. Nanoshells prepared by atomic layer deposition-long acting depots of indomethacin. Eur. J. Pharm. Biopharm. 2019, 140. 60-66.
(29) Zhang. D.; Quayle. M. J.; Petersson, G.; Van Ommen, J. R.; Folestad. S. Atomic scale surface engineering of micro- to nano-sized pharmaceutical particles for drug delivery applications. Nanoscale 2017, 9 (32). 11410-11417. DOI: 10.1039/c7nr03261g.
(30) La Zara, D.; Sun. F.; Zhang. F.; Franck. F.; Balogh Sivars. K.; Horndahl, J.; Bates, S.; Brännström, M.; Ewing. P.; Quayle. M. J.; Petersson, G.; Folestad. S.; Van Ommen. J. R. Controlled Pulmonary Delivery of Carrier-Free Budesonide Dry Powder by Atomic Layer Deposition. ACS Nano 2021, 15 (4). 6684-6698. DOI: 10.1021/acsnano.0c10040.
(31) Garcca. R. L.; Meinerz, N. M.; Dong. M.; Funke, H.; Ghazvini. S.; Randolph. T. W. Single-administration, thermostable human papillomavirus vaccines prepared with atomic layer deposition technology. NPJ Vaccines 2020, 5 (1), DOI: 10.1038/s41541-020-0195-4.
(32) Chazot. M.; Kostogiannes, A.; Julian, M.; Feit. C.; Sosa, J.; Kang. M.; Blanco, C.; Cook. J.; Rodriguez. V.; Adamictz. F. Enhancement of ZnSe stability during optical composite processing via atomic layer deposition. J. Non-Cryst. Solids 2022, 576. 121259.
(33) Adhikari. S.; Selvaraj. S.; Kim. D.-H. Progress in Powder Coating Technology Using Atomic Layer Deposition. Adv. Mater. Interfaces 2018, 5 (16), 1800581. DOI: 10.1002/admi.201800581.
(34) Acharjya. S. K.; Sahu. A.; Das. S.; Sagar. P.; Annapurna, M. M. Spectrophotometric methods for the determination of mesalamine in bulk and pharmaceutical dosage forms. Indian J. Pharm. Educ. Res. 2010, 1 (1). 63.
(35) Movahedinia, H. Investigation of 5-aminosalicylic acid (Mesalazine Drug) as a Corrosion Inhibitor for Carbon Steel in Sulfuric Acid Solution. Int. J. Electrochem. Sci. 2021, DOI: 10.20964/2021.02.29.
(36) Hu, D.; Liu, L.; Chen, W.; Li, S.; Zhao, Y. A novel preparation method for 5-aminosalicylic acid loaded Eudragit S100 nanoparticles. Int J Mol Sci 2012, 13 (5), 6454-68, DOI: 10.3390/ijms13056454.
(37) Dumitru, M. V.; Sandu, T.; Ciurlică, A. L.; Neblea, I. E.; Trică, B.; Ghebaur, A.; Gârca, S. A.; Iovu, H.; Sârbu, A.; Iordache, T. V. Organically modified montmorillonite as pH versatile carriers for delivery of 5-aminosalicylic acid. Appl. Clay Sci. 2022, 218, DOI: 10.1016/j.clay.2022.106415.
(38) Duan, H.; Lu, S.; Gao, C.; Bai, X.; Qin, H.; Wei, Y.; Wu, X.; Liu, M. Mucoadhesive microparticulates based on polysaccharide for target dual drug delivery of 5-aminosalicylic acid and curcumin to inflamed colon. Colloids Surf B Biointerfaces 2016, 145, 510-519, DOI: 10.1016/j.colsurfb.2016.05.038.
(39) Myers, T. J.; Cano, A. M.; Lancaster, D. K.; Clancey, J. W.; George, S. M. Conversion reactions in atomic layer processing with emphasis on ZnO conversion to Al₂O₃ by trimethylaluminum. J. Vac. Sci. Technol., A 2021, 39 (2), 021001, DOI: 10.1116/6.0000680.
(40) Lu, S.; Wang, H.; Zhou, J.; Wu, X.; Qin, W. Atomic layer deposition of ZnO on carbon black as nanostructured anode materials for high-performance lithium-ion batteries. Nanoscale 2017, 9 (3), 1184-1192, DOI: 10.1039/C6NR07868K.
(41) Boyadjiev, S. I.; Georgieva, V.; Yordanov, R.; Raicheva, Z.; Szilágyi, I. M. Preparation and characterization of ALD deposited ZnO thin films studied for gas sensors. Appl. Surf. Sci. 2016, 387, 1230-1235, DOI: https://doi.org/10.1016/j.apsusc.2016.06.007.
(42) Schmitt, H.; Creton, N.; Prashantha, K.; Soulestin, J.; Lacrampe, M. F.; Krawczak, P. Melt-blended halloysite nanotubes/wheat starch nanocomposites as drug delivery system. Polymer Engineering & Science 2015, 55 (3), 573-580, DOI: 10.1002/pen.23919.
(43) Pawar, A. R.; Mundhe, P. V.; Deshmukh, V. K.; Pandhare, R. B.; Nandgude, T. D. Enrichment of aqueous solubility and dissolution profile of mesalamine: In vitro evaluation of solid dispersion. J. Pharm. Biol. Sci. 2021, 9 (2), 127-135.
(44) Narayan Sahoo, R.; De, A.; Kataria, V.; Mallick, S. Solvent-free Hot Melt Extrusion Technique in Improving Mesalamine Release for Better Management of Inflammatory Bowel Disease. Indian J. Pharm. Educ. Res. 2019, 53 (4s), s554-s562, DOI: 10.5530/ijper.53.4s.150.
(45) Energy Table for EDS Analysis (Document ID: JEC6101C602A). www.jeol.com.
(46) George, S.; Ott, A.; Klaus, J. Surface chemistry for atomic layer growth. J. Phys. Chem. 1996, 100 (31), 13121-13131.
(47) Wu, F.; Banerjee, S.; Li, H.; Myung, Y.; Banerjee, P. Indirect Phase Transformation of CuO to Cu₂O on a Nanowire Surface. Langmuir 2016, 32 (18), 4485-4493, DOI: 10.1021/acs.langmuir.6b00915.
(48) Perez, I.; Robertson, E.; Banerjee, P.; Henn-Lecordier, L.; Son, S. J.; Lec, S. B.; Rubloff, G. W. TEM-based metrology for HfO2 layers and nanotubes formed in anodic aluminum oxide nanopore structures. Small 2008, 4 (8), 1223-1232.
(49) Gao, Z.; Wu, F.; Myung, Y.; Fei. R.; Kanjolia, R.; Yang, L.; Banerjee, P. Standing and Sitting Adlayers in Atomic Layer Deposition of ZnO. J. Vac. Sci. Technol., A 2016, 34 (1), 01A143.
(50) Guziewicz, E.; Kowalik, I. A.; Godlewski, M.; Kopalko, K.; Osinniy, V.; Wójcik, A.; Yatsunenko, S.; Łusakowska, E.; Paszkowicz, W.; Guziewicz, M. Extremely low temperature growth of ZnO by atomic layer deposition. J. Appl. Phys. 2008, 103 (3), 033515, DOI: 10.1063/1.2836819.
(51) Bacsa, J.; Hanke, F.; Hindley, S.; Odedra, R.; Darling, G. R.; Jones, A. C.; Steiner, A. The Solid-State Structures of Dimethylzinc and Diethylzinc. Angew. Chem. Int. Ed. 2011, 50 (49), 11685-11687, DOI: https://doi.org/10.1002/anie.201105099.
(52) Jensen, J. M.; Oelkers, A. B.; Toivola, R.; Johnson, D. C.; Elam, J. W.; George, S. M. X-ray reflectivity characterization of ZnO/Al₂O₃ multilayers prepared by atomic layer deposition. Chem. Mater. 2002, 14 (5), 2276-2282, DOI: 10.1021/cm011587z.
(53) Willis, S. A.; McGuinness, E. K.; Li, Y.; Losego, M. D. Re-examination of the Aqueous Stability of Atomic Layer Deposited (ALD) Amorphous Alumina (Al₂O₃) Thin Films and the Use of a Postdeposition Air Plasma Anneal to Enhance Stability. Langmuir 2021, 37 (49), 14509-14519, DOI: 10.1021/acs.langmuir.1c02574.
(54) Correa, G. C.; Bao, B.; Strandwitz, N. C. Chemical Stability of Titania and Alumina Thin Films Formed by Atomic Layer Deposition. ACS Appl. Mater. Interfaces 2015, 7 (27), 14816-14821, DOI: 10.1021/acsami.5b03278.
(55) Sun, K. G.; Li, Y. V.; Saint John, D. B.; Jackson, T. N. pH-Controlled Selective Etching of Al₂O₃ over ZnO. ACS Appl. Mater. Interfaces 2014, 6 (10), 7028-7031, DOI: 10.1021/am501912q.
(56) Kaur, H.; Kumar, S.; Kukkar, D.; Kaur, I.; Singh, K.; Bharadwaj, L. M. Transportation of drug-(polystyrene bead) conjugate by actomyosin motor system. J. Biomed. Nanotechnol. 2010, 6 (3), 279-286.
(57) Suhail, M.; Shao, Y. F.; Vu, Q. L.; Wu, P. C. Designing of pH-Sensitive Hydrogels for Colon Targeted Drug Delivery; Characterization and In Vitro Evaluation. GELS 2022, 8 (3), DOI: 10.3390/gels8030155.

### Supplemental References

Coile et al, J. Vac. Sci. Technol. A 38, 052403 (2020); doi: 10.1116/6.0000274
G. Socrates, Infrared and Raman characteristic group frequencies: tables and charts, John Wiley & Sons, 2004.
General information regarding ALD reactors was obtained from the website of the company Beneq on Feb. 9, 2023.
Teruel et al. Int. J. Mol. Sci. 2020, 21, 6502; doi: 10.3390/ijms21186502

What is claimed is:

1. A method for coating powder particles with metal oxide comprising:
loading a predetermined amount of the powder particles into a rotary barrel reactor of a rotary thermal atomic layer deposition (ALD) system;
activating a motor of the rotary thermal ALD system to rotate the rotary barrel reactor such that the powder particles are in motion when carrying out the method;
submitting the powder particles to at least one cycle of an ALD process, thereby coating the powder particles with the metal oxide to produce coated powder particles; and
unloading the coated powder particles from the rotary thermal ALD system upon completion of the at least one cycle of the ALD process.

2. The method according to claim 1, wherein the metal oxide is at least one of aluminium oxide (Al₂O₃) and zinc oxide (ZnO).

3. The method according to claim 1, wherein the powder particles are powder particles of an active pharmaceutical ingredient (API).

4. The method according to claim 3, wherein the active pharmaceutical ingredient (API) is 5-Aminosalicylic acid (5-ASA).

5. The method according to claim 1, wherein the at least one cycle of an ALD process comprises sequential steps of:
subjecting the powder particles to one pulse of a gaseous precursor metal;
subjecting the powder particles to a first purge of argon gas;
subjecting the powder particles to one pulse of deionized water; and
completing the at least one cycle of the ALD process by subjecting the powder particles to a second purge of the argon gas.

6. The method according to claim 5, wherein the at least one cycle of the ALD process is carried out at a temperature of less than 150° C.

7. The method according to claim 6, wherein the temperature of less than 150° C. is 120° C.

8. The method according to claim 5, wherein the gaseous precursor metal applied to the powder particles is at least one of trimethyl aluminium (TMA) and diethyl zinc (DEZ).

9. The method according to claim 5, wherein the one pulse of the gaseous precursor metal is applied to the powder particles for a time period of one second.

10. The method according to claim 5, wherein the one pulse of the deionized water is applied to the powder particles for a time period of a 0.5 second.

11. The method according to claim 5, wherein the first and second purges of argon gas are each 20 second flows of argon at 75 standard cubic centimetre per minute (sccm).

12. The method according to claim 5, wherein the at least one cycle of the ALD process is followed by additional sequential cycles of the ALD process.

13. The method according to claim 12, wherein an amount of the additional sequential cycles of the ALD process carried out is between one additional cycle and 300 additional cycles.

14. The method according to claim 13, wherein the amount of the additional sequential cycles of the ALD process carried out is 300 cycles.

15. The method according to claim 13, wherein the amount of the additional sequential cycles of the ALD process carried out is 200 cycles.

16. Powder particles having a coating of metal oxide prepared according to the method of claim 5.

17. The powder particles according to claim 16, wherein the coated powder particles are powder particles of 5-Aminosalicylic acid (5-ASA).

18. The powder particles according to claim 16, wherein the coating of metal oxide has a thickness corresponding to a thickness on a planar silicon substrate in a range of 30 to 33 nm.

19. A method for treating a gastrointestinal condition in a subject in need thereof, the method comprising:
providing a pharmaceutical composition including 5-Aminosalicylic acid (5-ASA) powder particles coated with a metal oxide; and
administering a pharmaceutically effective amount of the pharmaceutical composition to the subject, thereby treating the gastrointestinal condition in the subject.

20. The method according to claim 19, wherein the gastrointestinal condition is an inflammatory bowel disease (IBD).
